SEQUENCE LISTING (7 sequences)

SEQ ID NO: 1 - protein, 14 aa, synthetic construct
KLAKLAKKLAKLAK

SEQ ID NO: 2 - protein, 14 aa, synthetic construct
KLAKKLAKLAKKLA

SEQ ID NO: 3 - protein, 14 aa, synthetic construct
KAAKKAAKAAKKAA

SEQ ID NO: 4 - protein, 14 aa, synthetic construct
KLGKKLGKLGKKLG

SEQ ID NO: 5 - protein, 16 aa, synthetic construct
FQLFTPFSLGFEFRPG

SEQ ID NO: 6 - protein, 172 aa, synthetic construct
QDPGSKAVADRYAVYWNSSNPRFQRGDYHIDVCINDYLDVFCPHYEDSVPEDKTERYVLYMVNFDGYSACDHTSKGFKRWECNRPHSPNGPLKFSEKFQLFTPFSLGFEFRPGREYFYISSAIPDNGRRSCLKLKVFVRPTNSCMKTIGVHDRVFDVNDKVENSLEPADDTC

SEQ ID NO: 7 - DNA, 519 bp, synthetic construct
  source 1..519: mol_type = other DNA
caggacccgggctccaaggccgtcgccgaccgctacgctgtctactggaacagcagcaaccccagattccagaggggtgactaccacatcgacgtctgcatcaacgactacctggacgttttctgccctcactacgaggactccgtcccagaagacaagactgagcgctacgtcctctacatggtgaacttcgacggctactccgcctgcgaccacacttccaagggtttcaagagatgggaatgcaaccgtcctcactctccaaacggaccgctgaagttctctgaaaagttccagctcttcactcccttctctctgggattcgaattcaggccaggccgtgaatacttctacatctcctctgctatcccagacaacggaagaaggtcctgcctgaagctcaaggtcttcgtgagaccaacaaacagctgcatgaagactatcggtgttcacgaccgtgttttcgacgttaacgacaaggtggaaaactcactggaaccagctgacgacacctgctaa

(19) United States
(12) Patent Application Publication
(10) Pub. No.: US 2026/0295061 A1
(43) Pub. Date: Oct. 1, 2026

(54) MULTI-TARGET DRUG CONJUGATE AND USE THEREOF FOR CANCER TREATMENT

(71) Applicants: WAKE FOREST UNIVERSITY HEALTH SCIENCES, Winston-Salem, NC (US); VIRGINIA TECH INTELLECTUAL PROPERTIES, INC., Blacksburg, VA (US)

(72) Inventors: Waldemar DEBINSKI, Winston-Salem, NC (US); Denise HERPAI, Winston-Salem, NC (US); John H. ROSSMEISL, JR., Blacksburg, VA (US)

(21) Appl. No.: 19/479,406

(22) PCT Filed: May 2, 2024
(86) PCT No.: PCT/US2024/027428

§ 371 Date: Oct. 28, 2025

Related U.S. Application Data
(60) Provisional application No. 63/499,564, filed on May 2, 2023.
(60) Provisional application No. 63/582,616, filed on Sep. 14, 2023.

(51) Int. Cl. A61K 47/64; A61K 9/00; A61K 35/00; A61K 38/00; C07K 14/54; C07K 14/715; C12N 9/12; C12Q 1/48

(52) U.S. Cl. CPC A61K 47/642; A61K 9/0019; A61K 35/00; A61K 47/6425; C07K 14/5437; C07K 14/7155; C12N 9/12; C12Q 1/485; A61K 38/00; C07K 2319/30; C12Y 207/10001; G01N 2333/912

(57) ABSTRACT

Provided is a cancer targeting construct comprising a first end and a second end, the first end comprising an IL-13 targeting protein, or an IL-13RA2 binding mutant or fragment of IL-13, and the second end comprising an eA5 targeting protein, or an EphA2, EphA3 and EphB2 binding mutant or fragment of eA5, and an effector molecule coupled to said first end and/or said second end, wherein said construct is a fusion protein and/or a covalent conjugate. Also provided is a method of treating cancer (e.g., breast cancer) in a subject in need thereof, comprising administering to said subject a construct as taught herein, or composition comprising the construct, in a treatment effective amount.

### RELATED APPLICATIONS

This application claims the benefit of U.S. Provisional Patent Application No. 63/499,564, filed May 2, 2023, and U.S. Provisional Patent Application No. 63/582,616, filed Sep. 14, 2023, the disclosures of which are incorporated by reference herein.

### STATEMENT OF GOVERNMENT SUPPORT

This invention was made with government support under R01 CA256285, R01 CA276233, and 1P01 CA207206-01A1 awarded by the National Institutes of Health. The government has certain rights in the invention.

### STATEMENT REGARDING ELECTRONIC FILING OF A SEQUENCE LISTING

A Sequence Listing in XML format, entitled 9151-264WO_ST26.xml, 7,734 bytes in size, generated on Apr. 30, 2024, and filed herewith, is hereby incorporated by reference into the specification for its disclosures.

### BACKGROUND

Interleukin 13 receptor alpha 2 (IL-13RA2) and erythropoietin producing human hepatocellular (Eph) A2, A3 and B2 receptors are over-expressed in most patients with glioblastoma (GBM), but not in normal brain. Debinski et al. (1999) Clin Cancer Res 5:985-990; Mintz et al. (2002) Neoplasia 4:388-399; Debinski (1998) Crit Rev Oncog 9:255-268; Debinski et al. (2000) Mol Med 6:440-449. These receptors are also over-expressed in spontaneous canine glioma, the most faithful translational model of human disease. Debinski et al. PLOS One. 2013 Oct. 16; 8 (10): e77719. PMID: 24147065; Candolfi et al. J Neurooncol. 2007 November; 85 (2): 133-48; Dickinson et al. (2010) Neuro Oncol 12:928-940; Dickinson et al. (2008) J Neurosurg 108:989-998; Rossmeisl et al. Neuro Oncol. 2020 Aug. 19, PMID: 32812637.

Furthermore, IL-13RA2, EphA2, EphA3 and EphB2 are widely present in various compartments of GBM tumors. For example, all four receptors are expressed in tumor cells of the core and, importantly, infiltrating tumor cells, while EphA2 is also over-expressed in tumor neovasculature. Wykosky et al. Clin. Cancer Res. 14, 199-208 (2008); Ferluga et al. Oncotarget. 2016 Aug. 1. doi: 10.18632/oncotarget.10978. [Epub ahead of print] PMID: 27494882; Hatano et al. J Transl Med. 2004 Nov. 24; 2 (1): 40. PMID: 15563374; Brown et al. PLOS One. 2013 Oct. 18; 8 (10): e77769. eCollection 2013. PMID: 24204956.

Interestingly, IL-13RA2, EphA2, and EphA3 are associated with, and play crucial roles in, the pathobiology of glioma stem-like cells (GSC). IL-13RA2 is abundant in cells isolated as GSCs from GBM and contributes to their cell stem properties. EphA2 and EphA3 drive self-renewal and tumorigenicity of GSCs. Brown et al. (2013), supra; Brown et al. (2012) Clin Cancer Res 18:2199-2209; Brown et al. Cancer Res. 69 (23): 8886-93, 2009; Nguyen et al. (2011) Transl Oncol 4:390-400; Binda et al. Cancer Cell 22 (6): 765-80, 2012; Miao et al. Oncogene. 2014 Feb. 3. PMID: 24488013; Day et al. Cancer Cell 23 (2): 238-48, 2013. Finally, the EphA3 receptor can be readily detected in GBM-infiltrating cells of monocytic origin, glioma-associated macrophages (GAM). Ferluga et al. (2016), supra.

Thus, collectively, IL-13RA2, EphA2, EphA3 and EphB2 are expressed in several GBM compartments documented to be involved in tumor progression and/or resistance to therapies covering almost 100% of the tumor microenvironment. Sharma et al. Neuro-Oncology Advances, August 2020 vdaa107, https://doi.org/10.1093/noajnl/vdaa107; Sharma et al. Pharmaceuticals (Basel). 2020 Apr. 23; 13 (4): 77. doi: 10.3390/ph13040077. PMID: 32340173.

Despite these advances in our understanding of GBM tumors, treatment for GBM remains a significant unmet medical need. Despite nearly 80 years of effort, the two-year survival rate remains below 15%. Debinski et al. (1999), supra. More effective approaches for GBM treatment are greatly needed.

### SUMMARY

Provided herein according to some embodiments is a cancer targeting construct comprising a first end and a second end, the first end comprising an IL-13 targeting protein, or an IL-13RA2 binding mutant or fragment of IL-13, and the second end comprising an eA5 targeting protein, or an EphA2, EphA3 and EphB2 binding mutant or fragment of eA5, and an effector molecule coupled to said first end and/or said second end, wherein said construct is a fusion protein and/or a covalent conjugate.

In some embodiments, the construct comprises a first end and a second end, said first end having two IL-13 proteins (e.g., an IL-13 dimer), or two IL-13RA2 binding mutants or fragments of IL-13, said second end having two eA5 proteins (e.g., an eA5 dimer), or two EphA2, EphA3 and EphB2 binding mutants or fragments of eA5, and an effector molecule coupled to said first end or said second end, wherein said construct is a fusion protein and/or covalent conjugate, and wherein the effector molecule comprises a maytansinoid or a maytansinoid analog (e.g., DM1 or DM4).

In some embodiments, the two eA5 proteins are mutant eA5 proteins. In some embodiments, the two eA5 proteins are fragments of eA5 (e.g., amino acids 21-191 of human eA5).

In some embodiments, the two IL-13 proteins are mutant IL-13 proteins (e.g., IL-13.E13K).

In some embodiments, the eA5 and IL-13 proteins are glycosylated.

In some embodiments, the construct comprises a fusion protein with a protein linker between said first end and said second end. In some embodiments, the first end is at the N-terminus of the fusion protein, and the second end is at the C-terminus of the fusion protein. In some embodiments, the protein linker comprises an ADCC and/or CDC activating domain. In some embodiments, the protein linker comprises an Fc fragment of human IgG1.

In some embodiments, the effector molecule further comprises a detectable group.

Also provided is a composition comprising a construct as taught herein and a pharmaceutically acceptable carrier. In some embodiments, the carrier is sterile. In some embodiments, the carrier is a saline solution having a pH of from 6 or 6.5 to 7.7 or 8.

In some embodiments, the composition is suitable for administration to a subject by infusion (e.g., convection-enhanced delivery). In some embodiments, the composition is suitable for administration by continuous infusion of from 0.1 or 0.5 μg/mL to 20 μg/mL, for example, from 1, 2 or 3 μg/mL to 5, 8, 10 or 15 μg/mL.

Further provided is a nucleic acid that encodes a construct as taught herein, or a protein or peptide portion thereof. Still further provided is a eukaryotic host cell that contains such a nucleic acid is configured to express the encoded peptide. In some embodiments, the host cell is an insect expression cell.

Also provided is a method of treating cancer in a subject in need thereof, comprising administering to said subject a construct as taught herein, or composition comprising the construct, in a treatment effective amount.

In some embodiments, the cancer is breast cancer, bladder cancer, pancreatic cancer, colorectal cancer, head and neck cancer, thyroid cancer, prostate cancer, melanoma, or glioma. In some embodiments, the cancer is glioblastoma, prostate cancer or melanoma. In some embodiments, the cancer is glioblastoma.

In some embodiments, the administering comprises convention-enhanced delivery (CED). In some embodiments, the construct is administered by continuous infusion of from 0.1 or 0.5 μg/mL to 20 μg/mL, for example, from 1, 2 or 3 μg/mL to 5, 8, 10 or 15 μg/mL.

Further provided is a method of detecting EphA2, EphA3 and/or EphB2 expressing cells, comprising administering the construct as taught herein, or a composition comprising the construct, to a cell or group of cells, wherein said construct comprises a detectable group, and detecting said detectable group.

Further provided is a method of treating breast cancer (e.g., triple-negative breast cancer) in a subject in need thereof, comprising administering to said subject a construct as taught herein, or composition comprising the construct, in a treatment effective amount. In some embodiments, the construct is administered by intravenous injection. In some embodiments, the construct is administered in an amount from about 0.1 mg/kg to about 15 mg/kg, for example from 0.5, 1, 2, 3, 4, or 5 mg/kg, to 11, 12, 13, 14, or 15 mg/kg.

Also provided is the use of a construct as taught herein, or a composition comprising the construct, for treating cancer in a subject in need thereof, or for preparing a medicament for treating cancer in a subject in need thereof.

### BRIEF DESCRIPTION OF THE DRAWINGS

FIG. 1. Panel A) Schemata of IL-13M-(Fc)-eA5 conjugated to DM1-SMCC (QUAD-DM1). Panel B) Western blot of immunoreactivity of all main three elements of QUAD: IL-13.E13K, Fc, and ephrinA5.

FIG. 2. Exceptional responses to a cocktail of targeted cytotoxins in dogs with spontaneous gliomas. The best four responders out of 18 treated dogs with >95% volumetric tumor regression in a trial using DT390-IL-13M and eA1M-PE38QQR; 17 animals have been enrolled and dose limiting toxicity was not seen. Tumor regression was even more pronounced at 16 weeks post treatment compared to 6 weeks. Dog 141004 had PD at 16 weeks post-CED.

FIG. 3. Comparative gene expression analyses of canine gliomas before and after treatment with a cocktail of targeted cytotoxins against IL-13RA2 and EphA2 receptors. Panel A) Ingenuity Pathway Analyses of the top ranked signaling pathways altered by the cytotoxin treatment based on z-score. Panel B) Increased expression of the Th1 response genes IL2 and IFNG in the post-treatment tumors of responder cases. Panel C) Pooled DEG datasets from the 3 dogs were compared using GSEA incorporating cell type signatures for infiltrating immune cell populations. Post-treatment samples demonstrated increases in activated helper (CD4+) and cytotoxic T-lymphocytes (CD8+), NK cells, and glioma-infiltrating macrophages and microglia (GIM/Ms). D) Immunofluorescent staining of CD4+ and CD8+ lymphocytes in tumor samples, Dog 4.

FIG. 4. Schemata of QUAD 1.0 and 3.0. The arrangement for QUAD 1.0 is eA5-CH2CH3-IL-13M, and for QUAD 3.0 is IL-13M-CH2CH3-eA5 plus cysteine at the C-terminal end of the protein chains. The cysteine serves as a specific conjugation site to drugs/toxins.

FIG. 5. Imaging based canine tumor responses following treatment with QUAD-DOX (Dog 2; top row) and QUAD-PE38QQR (Dog 5; middle and bottom rows). In Dog 2, there is serial decrease in tumor volume with the best post-treatment response occurring 6 months after treatment. In Dog 5, pseudoprogression was observed at 6 weeks, with subsequent elimination of all enhancing lesions and a 91% reduction in the T2/FLAIR lesion volume by 6 months after treatment.

FIG. 6. Human eA5 interacts with the receptors in a cross-species manner. Flow cytometry of QUAD 3.0 binding to canine G06-A GBM cell line (Panel A), and potent killing of these cells by QUAD 3.0-PE38QQR (Panel B). Down-regulation of the EphA2 and EphA3 receptors by human eA5 (1 g/ml) (R&D Systems) in two canine GBM cell lines: G06-A and SDT-3G (Panel C). These results mimic exactly what we found in human GBM cells. Panel D) Activation of phospho-EphA3 by human eA5-Fc in two canine GBM cell lines: G06-A and SDT-3G. Panel E) Down-regulation of the EphA2 and Eph3A receptors in a mouse glioma GL261 cells (IL-13RA2+). Panel F) ELISA of the binding of human eA5 to recombinant murine and human EphA3 receptors. Note that the human eA5-Fc binds the murine EphA3 receptor slightly better than the human EphA3 receptor. Densitometry values of fold change are shown in immunoblots.

FIG. 7. Panel A) The structure of DM1 (microtubule disrupting agent) that is also thiol-reactive and form a stable thioether bond with the thiol present in the C-terminal cysteine residues in QUAD; Panel B) SDS-PAGE of QUAD 3.1 (lane 1) and its QUAD-DMI conjugate (lane 2); Panel C) Cell viability assay of QUAD 3.1 conjugates: two different batches of QUAD-DMI: #1 and #2, QUAD-DTX (deruxtecan) and QUAD-WP936 (doxorubicin) conjugates, plus unconjugated DM1-SMCC on U-251 MG GBM cells; Panel D) Cell viability assay of QUAD-DMI conjugate on human U-251 and canine G06-A and SDT-3G GBM cells. Panel E) Cell viability assay on U-251 and T98G GBM cells. T98G are IL-13RA2 (−) and non-responders to IL13-based cytotoxins. Panel F) Unconjugated QUAD (100 nM) neutralizes cell killing activity of QUADDM1 on U-251 GBM cells. Panel G) Unconjugated QUAD possesses some intrinsic anti-tumor activity. Panels H, I) Toxicity study of QUAD-DMI in mice given the conjugate either intracranially (IC; Panel H) or intravenously (IV; Panel I). Three mice were used per dose.

FIG. 8. Panels A-D) Pre-treatment T2W MRI of high-grade oligodendroglioma in left frontoparietal lobe. Panels E-H) Intraoperative 3DT1W MRI monitored CED infusion of QUAD-DM1 (Vi=837 μl; [3.2 μg/ml]) demonstrating 92% coverage of the T2W lesion burden. Panels I-L) 8 weeks post-treatment T2W MRI with partial tumor response characterized by a striking 93% reduction in tumor volume.

FIG. 9. Panel A) Heatmap showing gene expression of EphA2, EphA3, EphB2, and IL13RA2 based on samples, in whole or in part, from data generated by the TCGA Research Network (cancer.gov/tcga); Panel B) western blot of EphA2, EphA3, EphB2, and β-actin control in various breast cancer cell lines; Panel C) flow cytometry of QUAD binding to human breast cancer cell lines.

FIG. 10. Panels A-D) Immunohistochemistry staining for IL-13RA2, EphA3, EphA2, and EphB2 in tissue microarrays of breast cancer, lymph node metastasis, and normal breast; isotype controls (Jackson Immuno) for each stain are as indicated in the right panels; rows 1-5 are breast cancer; rows 6-9 are involved lymph nodes; row 10 is adjacent, normal breast tissue.

FIG. 11. Immunohistochemistry staining for IL-13RA2, EphA3, EphA2, and EphB2 in primary breast cancer and subsequent brain metastasis from the same patients; Patient #1 had triple-negative breast cancer and Patient #2 had triple-positive breast cancer.

FIG. 12. Panel A) Cell viability assay of QUAD-DMI conjugate on GBM cells (U-251) and seven breast cancer cell lines, including two pairs of primary and metastatic tumors (MDA-468 and MDA-231); Panel B) flow cytometry data of propidium iodide nuclei staining showing the percentage of cells in each growth phase after treatment with 1 nM QUAD-DMI; Panel C) MDA-MB-231 tumor volume measurements in nude mice over 24 days after treatment with either IV injections of 12 mg/kg QUAD-DMI or PBS as a control.

FIG. 13. Intracranial (IC) treatment of MDA-MB-231-BrM tumors with QUAD-DM1, 200,000 MDA-MB-231-BrM cells in 2 μL-IC implantation. Treatment started 7 days post tumor cells implantation (Day 0). Animals were imaged 3 times per week vis IVIS imaging with bio-luminescence (photons per sec).

FIG. 14. Binding specificity (ELISA assay) of HeK-293 cells-produced QUAD to: EphA3, PDL1, and CD80. QUAD binds only to the EphA3 receptor as expected. Hek-293 cells are human embryonic kidney cells. The same result was obtained with QUAD produced in Expi-293F cells that are HEK-293 cells adapted for transient transfection.

FIG. 15. MDA-MB-231 BrM cell killing by QUAD-DMI read at 48 and 72 hrs after adding QUAD-DM1. The cell killing by QUAD-DMI was partially blocked by the antibodies against individual receptor binding abilities of the QUAD ligand.

FIG. 16. HC1806—triple negative breast cancer cells (TNBC), with methods similar to that of the data in FIG. 15.

### DETAILED DESCRIPTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the invention. Hence, the following specification is intended to illustrate some particular embodiments of the invention, but not to exhaustively specify all permutations, combinations and variations thereof.

The disclosures of all United States patent references cited herein are to be incorporated herein by reference to the extent they are consistent with the present disclosure.

Unless otherwise defined, all technical and scientific terms used herein have the same meaning as commonly understood by one of skill in the art to which this invention belongs. The terminology used in the description of the invention herein is for the purpose of describing particular embodiments only and is not intended to be limiting of the invention.

Except as otherwise indicated, standard methods may be used for cloning genes, amplifying and detecting nucleic acids, and the like. Such techniques are known to those skilled in the art. See, e.g., Sambrook et al., Molecular Cloning: A Laboratory Manual 4th Ed. (Cold Spring Harbor, NY, 2012); Ausubel et al. Current Protocols in Molecular Biology (Green Publishing Associates, Inc. and John Wiley & Sons, Inc., New York).

As used in the description of the invention and the appended claims, the singular forms “a,” “an,” and “the” are intended to include the plural forms as well, unless the context clearly indicates otherwise. Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of polypeptide, dose, time, temperature, enzymatic activity or other biological activity and the like, is meant to encompass variations of 10%, 5%, 1%, 0.5%, or even 0.1% of the specified amount.

Provided herein are constructs comprising a first end having one or two eA5 proteins, or a EphA2, EphA3 and EphB2 binding mutant or fragment thereof, and a second end having one or two IL-13 proteins, or an IL-13RA2 binding mutant or fragment thereof. See, e.g., Sharma et al., Neuro-Oncology Advances 2 (1), 1-11, 2020; Sharma et al., Pharmaceuticals 13, 77, 2020; and U.S. Pat. No. 9,975,942 to Debinski et al., which are incorporated by reference herein. In some embodiments, the construct comprises a human eA5 protein or EphA2, EphA3 and EphB2 binding mutant or fragment thereof. In some embodiments, the construct comprises a human IL-13 protein, or an IL-13RA2 binding mutant or fragment thereof.

The terms “peptide,” “polypeptide,” and “protein” are used interchangeably and refer to any polymer of amino acids (dipeptide or greater) linked through peptide bonds.

“Recombinant” nucleic acid as used herein refers to a nucleic acid produced in vitro, e.g., by synthesis and/or by combining two or more nucleic acid sequences from different sources (e.g., a “heterologous” nucleic acid). The recombinant nucleic acid may be provided in the form of a “vector” or “delivery vector” in order to transform or transfect cells to contain the nucleic acid. As used herein, a “vector” or “delivery vector” can be a viral or non-viral vector that is used to deliver a nucleic acid to a cell, tissue or subject.

A “recombinant” protein is a protein produced by a recombinant nucleic acid, often with the use of host cells. The nucleic acid may or may not be inserted into the genome of a host cell. The nucleic acid may exist, e.g., in plasmid form in a host cell. Alternatively, the recombinant protein may be produced by in vitro translation of the recombinant nucleic acid.

An “isolated” protein or polypeptide means a protein or polypeptide that is separated or substantially free from at least some of the other components of the naturally occurring organism or virus, for example, the cell or viral structural components or other proteins or nucleic acids commonly found associated with the protein. As used herein, the “isolated” protein or polypeptide is at least about 25%, 30%, 40%, 50%, 60%, 70%, 75%, 80%, 85%, 90%, 95%, 97%, 98%, 99% or more pure (w/w).

“Subjects” as used herein are generally human subjects and include, but are not limited to, cancer patients. The subjects may be male or female and may be of any race or ethnicity. The subjects may be of any age, including newborn, neonate, infant, child, adolescent, adult, and geriatric. Subjects may also include animal subjects, particularly mammalian subjects such as canines, felines, bovines, caprines, equines, ovines, porcines, rodents (e.g. rats and mice), lagomorphs, primates (including non-human primates), etc., such as for veterinary medicine or pharmaceutical drug development purposes.

“Cancer” or “cancers” that can be detected and/or treated by the constructs, compositions and methods described herein include, but are not limited to, breast cancer, bladder cancer, pancreatic cancer, colorectal cancer, head and neck cancer, thyroid cancer, prostate cancer, melanoma, and brain cancer such as gliomas (e.g., GBM), etc.

Many cancers over-express IL-13RA2 (e.g., GBM and other brain cancers, human pediatric brain tumors, brainstem glioma, renal cell carcinoma, squamous cell carcinoma of head and neck, ovarian cell carcinoma, pancreatic cancer, colorectal cancer, and melanoma), EphA2 (e.g., GBM and other brain cancer, breast cancer, prostate cancer, urinary bladder cancer, skin cancer, lung cancer, ovarian cancer, esophageal cancer, renal cancer, colon cancer and vulvar cancer), EphA3 (e.g., GBM and other brain cancers, leukemia, lymphoma, lung cancer, skin cancer and gastric carcinoma) and/or EphB2 (e.g., GBM and other brain cancers, gastric cancer, colon cancer, neuroblastomas, small cell lung carcinoma, and melanoma). This expression may have concomitant presence in various tumor compartments.

“Brain cancer” or “brain tumor” may be of any stage, grade, histomorphological feature, invasiveness, aggressivity or malignancy of an affected tissue or cell aggregation in any part of the central nervous system (i.e., brain and spinal cord). In some embodiments, the brain tumor is a glioma. In some embodiments, the tumor is an anaplastic astrocytoma, anaplastic oligoastrocytoma or anaplastic oligodendroglioma, in particular, fibrillary astrocytoma WHO grade II, oligoastrocytoma WHO grade II, oligodendroglioma grade II, anaplastic astrocytoma WHO grade III, anaplastic oligoastrocytoma WHO grade III, anaplastic oligodendroglioma grade III or glioblastoma multiforme (see, e.g., US Patent Application Publication No. 2010/0291590).

Gliomas are tumors occurring in the glial cells, which help support and protect critical areas of the brain. Gliomas are the most common type of brain tumor in adults, responsible for about 42% of all adult brain tumors. Gliomas are further characterized by the types of cells they affect, into the categories of astrocytoma (affecting astrocytes), oligodendroglioma (affecting oligodendrocytes), ependymoma (affecting ependymal cells), meningiomas (affecting the meninges), acoustic neuroma/schwannoma (affecting Schwann's cells), and medulloblastoma (affecting cells in the cerebellum). See also U.S. 2013/0012452 to Basile et al.

Astrocytomas are graded from I to IV depending on the speed of progression. Grade I (pilocytic astrocytoma) is slow growing, with little tendency to infiltrate surrounding brain tissue. Grade II (diffuse astrocytoma) is fairly slow-growing, with some tendency to infiltrate surrounding brain tissue. Grade III (anaplastic/malignant astrocytoma) tumors grow rather quickly and infiltrate surrounding brain tissue. Grade IV (glioblastoma or “GBM”) is an extremely aggressive and lethal form of brain cancer. Unfortunately, it is the most common form of brain tumor in adults, accounting for about 67% of all astrocytomas.

Oligodendrogliomas, which make up 4% of brain tumors, mostly affect people over 45 years of age. Some subtypes of this tumor are particularly sensitive to treatment with radiation therapy and chemotherapy. Half of patients with oligodendrogliomas are still alive after five years.

Ependymomas are rare; about 2% of all brain tumors, but are the most common brain tumor in children. They generally do not affect healthy brain tissue and do not spread beyond the ependyma. Although these tumors respond well to surgery, particularly those on the spine, ependymomas cannot always be completely removed. The five-year survival rate for patients over age 45 approaches 70%.

Meningiomas affect the meninges, the tissue that forms the protective outer covering of the brain and spine. One-quarter of all brain and spinal tumors are meningiomas, and up to 85% of them are benign.

Malignant gliomas are a fatal disease with an average life-expectancy following diagnosis of less than one year. The prognosis for patients with high-grade gliomas is very poor, and is especially so for older patients. Of Americans diagnosed each year with malignant gliomas, about half are alive 1 year after diagnosis, and 25% after two years. Those with anaplastic astrocytoma survive about three years. Glioblastoma has the worst prognosis, with a life expectancy of less than 9-15 months following diagnosis.

“Breast cancer” or “breast tumor” may be of any stage, grade, histomorphological feature, invasiveness, aggressivity or malignancy of an affected tissue or cell aggregation in any part of the breast tissue. In some embodiments, the breast cancer is inflammatory breast cancer or Paget's disease of the breast. In some embodiments, the breast tumor is a ductal carcinoma (invasive or non-invasive), a lobular carcinoma (invasive or non-invasive), an angiosarcoma, an adenocarcinoma, and/or a Phyllodes tumor. In some embodiments, the breast cancer is characterized as human epidermal growth factor 2 (HER2) positive (HER2+). In some embodiments, the breast cancer is characterized as estrogen receptor-negative (ER−), progesterone receptor-negative (PR−), and/or HER2-negative (HER2−).

In some embodiments, the breast cancer is ER−, PR−, and HER2− (e.g., triple-negative breast cancer (TNBC)). In some embodiments, the breast cancer and/or breast tumor expresses IL-13RA2, EphA2, EphA3, and/or EphB2. In some embodiments, the breast cancer and/or breast tumor over-expresses IL-13RA2, EphA2, EphA3, and/or EphB2.

In some embodiments, the breast cancer is a metastatic breast cancer. In some embodiments, the metastatic breast cancer metastasizes to the bones, lungs, brain, and/or liver. In some embodiments, the metastatic breast cancer metastasizes to more than one site in the subject (e.g., about 1, 2, 3, 4, 5, or more sites). In some embodiments, the metastasis site of the metastatic breast cancer is treated the same or differently from the primary tumor (i.e., using the chemotherapeutic and/or administration method, or a different chemotherapeutic and/or administration method).

“Effector molecule” as used herein includes therapeutic agents, nanoparticles, detectable groups, targeting ligands, and delivery vehicles (e.g., antibodies, lipids, liposomes). See, e.g., U.S. Pat. No. 6,630,576.

“Therapeutic agent” as used herein may be any therapeutic agent including, but not limited to, genetic materials or agents, radionuclides, chemotherapeutic agents, cytotoxic agents (see, e.g., U.S. Pat. No. 6,949,245 to Sliwkowski), and amphipathic antimicrobial peptides. Other exemplary therapeutic agents include, but are not limited to, radiopharmaceuticals, including, but not limited to Auger electrons, chemotherapeutic agents incorporating a radionuclide, and photosensitizers.

“Radionuclide” as described herein includes, but is not limited to, ²²⁷ Ac, ²¹¹ At, ¹³¹Ba, ⁷⁷Br, ¹⁰⁹Cd, ⁵¹Cr, ⁶⁷Cu, ¹⁶⁵Dy, ¹⁵⁵Eu, ¹⁵³Gd, ¹⁹⁸ Au, ¹⁶⁶Ho, ^{113m}In, ^{115m}In, ¹²³I, ¹²⁵I, ¹³¹I, ¹⁸⁹Ir, ¹⁹¹Ir, ¹⁹²Ir, ¹⁹⁴Ir, ⁵²Fe, ⁵⁵Fe, ⁵⁹Fe, ¹⁷⁷Lu, ¹⁰⁹Pd, ³²p, ²²⁶Ra, ¹⁸⁶Re, ¹⁸⁸Re, ¹⁵³Sm, ⁴⁶Sc, ⁴⁷Sc, ⁷²Se, ⁷⁵Se, ¹⁰⁵ Ag, ⁸⁹Sr, ³⁵S, ¹⁷⁷Ta, ^{117m}Sn, ¹²¹Sn, ¹⁶⁶Yb, ¹⁶⁹Yb, ⁹⁰Y, ²¹²Bi, ¹¹⁹Sb, ¹⁹⁷Hg, ⁹⁷Ru, ¹⁰⁰Pd, ^{101m}Rh, and ²¹²Pb.

“Chemotherapeutic agent” as used herein includes, but is not limited to, methotrexate, daunomycin, mitomycin C, cisplatin, vincristine, epirubicin, fluorouracil, verapamil, cyclophosphamide, cytosine arabinoside, aminopterin, bleomycin, mitomycin C, democolcine, etoposide, mithramycin, chlorambucil, melphalan, daunorubicin, doxorubicin, tamosifen, paclitaxel, vincristin, vinblastine, camptothecin, actinomycin D, and cytarabine. Other examples are found in U.S. Patent Application Publication 2006/0121539 (Debinski et al.), which is incorporated by reference herein in its entirety. Example chemotherapeutic agents include topoisomerase I inhibitors, such as camptothecins (e.g., topotecan and irinotecan) and indenoisoquinolines (e.g., indotecan and indimitecan), Deruxtecan (DTX), etc.

“Cytotoxic agent” or “toxic agent” as used herein includes, but is not limited to, maytansinoids and maytansinoid analogs, taxoids, CC-1065 and CC-1065 analogs, dolastatin and dolastatin analogs, ricin (or more particularly the ricin A chain), aclacinomycin, Diphtheria toxin, Monensin, Verrucarin A, Abrin, Tricothecenes, and Pseudomonas exotoxin A, taxol, cytochalasin B, gramicidin D, ethidium bromide, emetine, mitomycin, etoposide, tenoposide, anti-mitotic agents, such as the vinca alkaloids (e.g., vincristine and vinblastine), colchicin, anthracyclines, such as doxorubicin (inclusive of 4′-O-benzylated Dox analogs WP744 and WP769, or Dox analogs WP1244, WP936 and WP1737) and daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof, antimetabolites (e.g., methotrexate, 6-mercaptopurine, 6-thioguanine, cytarabine, and 5-fluorouracil decarbazine), alkylating agents (e.g., mechlorethamine, thioepa chlorambucil, melphalan, carmustine (BSNU), lomustine (CCNU), cyclothosphamide, busulfan, dibromomannitol, streptozotocin, mitomycin C, and cis-dichlorodiamine platinum (II) (DDP)), and antibiotics, including, but not limited to, dactinomycin (formerly actinomycin), bleomycin, mithramycin, calicheamicin, and anthramycin (AMC).

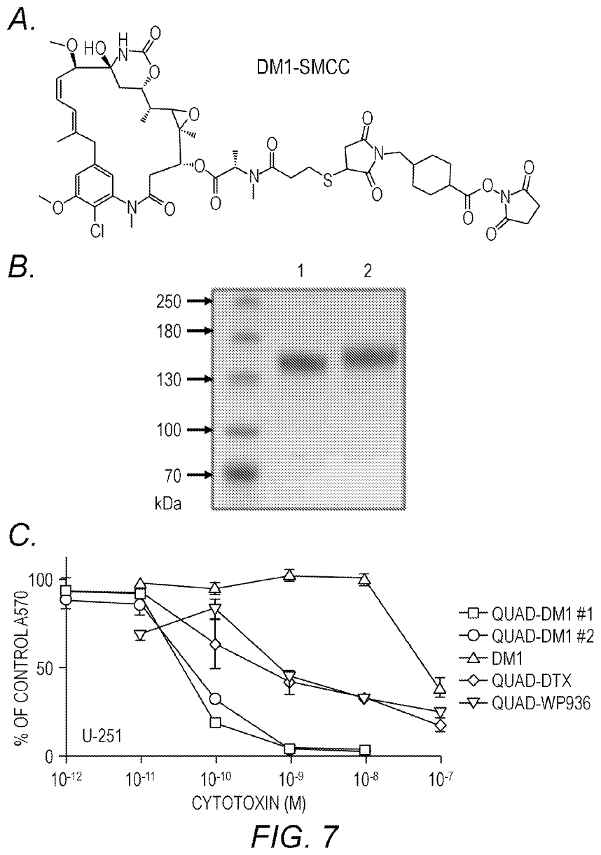

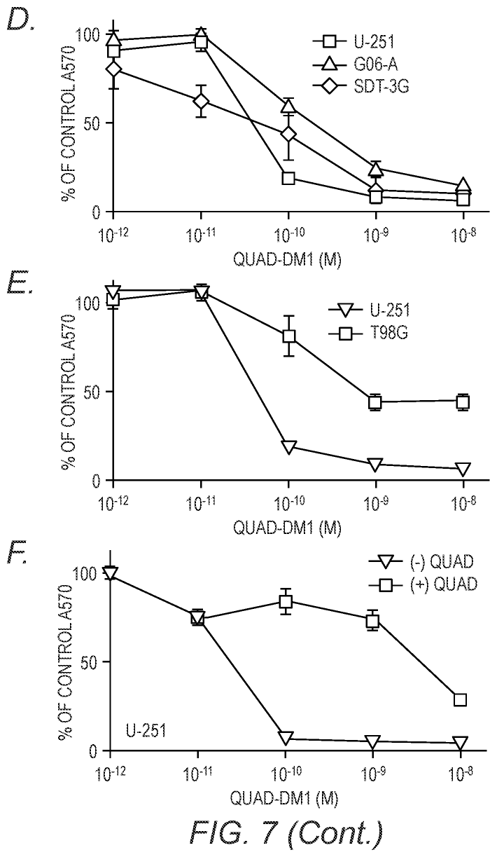

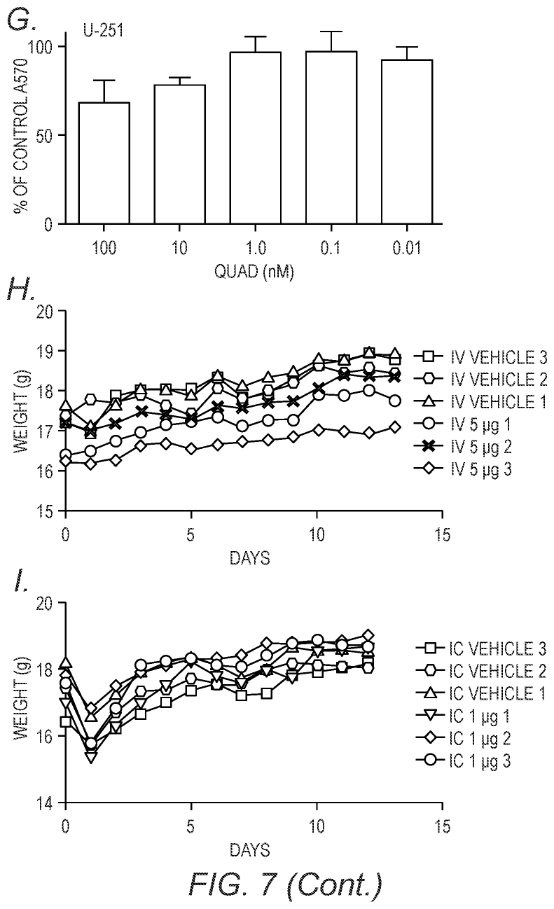

In some preferred embodiments, the cytotoxic agent is a microtubulin polymerization inhibitor. Particular examples include, but are not limited to, a benzoansamacrolide such as maytansine, or a derivative thereof, or a maytansinoid or a maytansinoid analog. Examples of such agents include, but are not limited to, drug maytansinoids (DMs) such as DM1, DM4, etc. Protein conjugates of maytansinoids are known. See, e.g., U.S. Pat. No. 8,624,003 to Kellogg et al., which is incorporated by reference herein. As shown, for example, in FIG. 7, Panel C herein, QUAD with DMI shows a notably high cytotoxic potency compared to the use of other cytotoxic agents.

In some embodiments, the maytansinoid is conjugated via a disulfide linker (e.g. a succinimidyl 4-(N-maleimidomethyl)cyclohexane-1-carboxylate (SMCC) linker). See, e.g., U.S. Pat. Nos. 5,208,020, 5,416,064, 6,441,163, and U.S. Patent Publication No. 2007/0048314.

In some embodiments, cytotoxic agents include toxins such as Pseudomonas exotoxin, ricin, abrin, ribonuclease (RNase), DNase I, Staphylococcal enterotoxin-A, pokeweed antiviral protein, gelonin, diphtheria toxin, etc. See, e.g., U.S. Pat. No. 7,517,964. In some embodiments, Pseudomonas exotoxin or a Diphtheria toxin is preferred. See U.S. Pat. No. 5,328,984 to Pastan et al. and U.S. Pat. No. 6,296,843 to Debinski, which are each incorporated by reference herein in its entirety. Pseudomonas exotoxins can include, but are not limited to, Pseudomonas exotoxin A (PE). The Pseudomonas exotoxin can be modified such that it substantially lacks domain Ia, and in some embodiments Pseudomonas exotoxins include PE38QQR and PE4E. Diphtheria toxins can include DT390, a diphtheria toxin in which the native binding domain is eliminated. It will be appreciated that in various embodiments, the therapeutic agents can be attached to, e.g., the amino terminus or the carboxyl terminus.

“Amphipathic antimicrobial peptide” as used herein includes amphipathic peptides that induce apoptosis of cancer cells, presumably through their ability to depolarize mitochondrial membranes. K. Rege et al., Cancer Res. 67, 6368 (2007). Such peptides are, in general, from 10, 12 or 13 to 20, 30 or 40 amino acids in length, or more, and typically have an amphipathic alpha-helical structure. Examples include, but are not limited to, (KLAKLAK)₂ (SEQ ID NO: 1); (KLAKKLA)₂ (SEQ ID NO:2) (KAAKKAA)₂ (SEQ ID NO:3) and (KLGKKLG)₂ (SEQ ID NO:4). See. e.g., Ruoslahti et al., U.S. Patent Application Publication No. 2001/0046498 (Nov. 29, 2001).

“Nanoparticle” as used herein includes particles that are about 0.5 to about 1,000 nanometers in size and may include natural and/or synthetic moieties. In some embodiments, the nanoparticle crosses the blood brain barrier. In some embodiments, the nanoparticle may incorporate a therapeutic agent. See, e.g., U.S. Pat. No. 8,535,726 to Dai et al.; U.S. Pat. No. 8,252,338 to Forte et al.; U.S. Pat. No. 8,246,968 to Zale et al.; U.S. 2013/0122056 to Zhang et al. In some embodiments, the nanoparticle comprises a polymeric matrix, which may comprise two or more polymers. One or more polymers of the polymeric matrix may include, e.g., polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, or combinations thereof. In some embodiments, the polymeric matrix comprises one or more polyesters, polyanhydrides, polyethers, polyurethanes, polymethacrylates, polyacrylates or polycyanoacrylates. In some embodiments, at least one polymer is a polyalkylene glycol. In some embodiments, the polyalkylene glycol is polyethylene glycol. In some embodiments, at least one polymer is a polyester. In some embodiments, the polyester is selected from the group consisting of PLGA, PLA, PGA, and polycaprolactones. In some embodiments, the polyester is PLGA or PLA. In some embodiments, the polymeric matrix comprises a copolymer of two or more polymers, such as a copolymer of a polyalkylene glycol and a polyester. In some embodiments, the copolymer is a copolymer of PLGA or PLA and PEG. In some embodiments, the polymeric matrix comprises PLGA or PLA and a copolymer of PLGA or PLA and PEG.

“Detectable group” or “label” as used herein includes, but is not limited to, radiolabels (e.g., ³⁵S, ¹²⁵I, ³²P, ³H, ¹⁴C, ¹³¹I), enzyme labels (e.g., horseradish peroxidase, alkaline phosphatase), gold beads, chemiluminescence labels, ligands (e.g., biotin, digoxin) and/or fluorescence labels (e.g., rhodamine, phycoerythrin, fluorescein, fluorescent proteins), a fluorescent protein including, but not limited to, a green fluorescent protein or one of its many modified forms, a nucleic acid segment in accordance with known techniques, and energy absorbing and energy emitting agents. Thus “label” or “detectable group” as used herein may be any suitable label or detectable group detectable by spectroscopic, photochemical, biochemical, immunochemical, electrical, optical or chemical means, including, but not limited to, biotin, fluorophores, antigens, porphyrins, and radioactive isotopes. Labels useful in the present invention include biotin for staining with labeled avidin or streptavidin conjugate, magnetic beads (e.g., Dynabeads TM), fluorescent dyes (e.g., fluorescein, fluorescein-isothiocyanate [FITC], Texas red, rhodamine, green fluorescent protein, enhanced green fluorescent protein, lissamine, phycoerythrin, Cy2, Cy3, Cy3.5, Cy5, Cy5.5, Cy7, FluorX [Amersham], SyBR Green I & II [Molecular Probes], and the like), radiolabels (e.g., ³H, ³⁵S, ¹⁴C, or ³²P), enzymes (e.g., hydrolases, particularly phosphatases such as alkaline phosphatase, esterases and glycosidases, or oxidoreductases, particularly peroxidases such as horseradish peroxidase, and the like), substrates, cofactors, inhibitors, chemiluminescent groups, chromogenic agents, and calorimetric labels such as colloidal gold or colored glass or plastic (e.g., polystyrene, polypropylene, latex, etc.) beads.

“Treat,” “treating” or “treatment” as used herein refers to any type of treatment that imparts a benefit to a subject afflicted with a disease, including improvement in the condition of the subject (e.g., in one or more symptoms), delay in the progression of the disease, and may include reduction in tumor volume or invasiveness, lengthening of average life expectancy, etc.

“Pharmaceutically acceptable” as used herein means that the construct or composition is suitable for administration to a subject to achieve the treatments described herein, without unduly deleterious side effects in light of the severity of the disease and necessity of the treatment.

In some embodiments, the constructs are fusion protein and/or covalent conjugate constructs, said constructs comprising a first end having two eA5 proteins (eA5 dimer), or a EphA2, EphA3 and EphB2 binding mutant or fragment thereof, and a second end having two IL-13 proteins (IL-13 dimer), or an IL-13RA2 binding mutant or fragment thereof. Dimers of the proteins may be formed using methods known in the art, e.g., with an Fc fusion protein or hinge linker. See, e.g., U.S. 2010/0209424 to Roopenian et al. and U.S. 2012/0039880 to Yan et al., which are incorporated by reference herein.

In some embodiments, the construct includes a glycosylated form of eA5, mutants or fragments thereof. See Ferluga et al., J Biol Chem 288 (25): 18448-18457 (2013). In some embodiments, the construct includes a glycosylated form of IL-13, mutants or fragments thereof.

In some embodiments, the eA5 mutant is a G-H loop mutant, such as ¹¹⁸FQLFTPFSLGFEFRPG¹³³ (SEQ ID NO:5) of eA5 (UniProtKB/Swiss-Prot Accession No. P52803.1). See Lema Tome et al., J Biol Chem 287:14012-14022 (2012). In some embodiments, the mutation is at amino acid 119 (Q), 123 (P), 125 (S), 127 (G), 132 (P), or any combination thereof, of eA5. In some embodiments, the mutation is at P123, S125, G127 or P132 of eA5, and the wild-type amino acid is substituted with A. In some embodiments, the eA5 mutant may have more than one, two or even three or more, amino acid changes. In some embodiments, the eA5 mutant has an enhanced binding affinity for EphA2, EphA3 and/or EphB2, as compared to the corresponding wild-type eA5 binding affinity.

As a non-limiting example of an eA5 fragment that may be used as an EphA2, EphA3, and EphB2 binding fragment in accordance with the present invention, the eA5 fragment may consist of amino acids 21-191 of human eA5, with a cysteine at the end (underlined) allowing for active agent conjugation, as set forth in SEQ ID NO:6:

QDPGSKAVADRYAVYWNSSNPRFQRGDYHIDVCINDYLDVFCPHYEDSV
PEDKTERYVLYMVNFDGYSACDHTSKGFKRWECNRPHSPNGPLKFSEKF
QLFTPFSLGFEFRPGREYFYISSAIPDNGRRSCLKLKVFVRPTNSCMKT
IGVHDRVFDVNDKVENSLEPADDTC

A non-limiting example of an optimized DNA sequence that may be used to encode the eA5 fragment provided as SEQ ID NO:6 is that of SEQ ID NO:7:

CAGGACCCGGGCTCCAAGGCCGTCGCCGACCGCTACGCTGTCTACTGGA
ACAGCAGCAACCCCAGATTCCAGAGGGGTGACTACCACATCGACGTCTG
CATCAACGACTACCTGGACGTTTTCTGCCCTCACTACGAGGACTCCGTC
CCAGAAGACAAGACTGAGCGCTACGTCCTCTACATGGTGAACTTCGACG
GCTACTCCGCCTGCGACCACACTTCCAAGGGTTTCAAGAGATGGGAATG
CAACCGTCCTCACTCTCCAAACGGACCGCTGAAGTTCTCTGAAAAGTTC
CAGCTCTTCACTCCCTTCTCTCTGGGATTCGAATTCAGGCCAGGCCGTG
AATACTTCTACATCTCCTCTGCTATCCCAGACAACGGAAGAAGGTCCTG
CCTGAAGCTCAAGGTCTTCGTGAGACCAACAAACAGCTGCATGAAGACT
ATCGGTGTTCACGACCGTGTTTTCGACGTTAACGACAAGGTGGAAAACT
CACTGGAACCAGCTGACGACACCTGCTAA

The targeting peptides of the present invention can be coupled to or conjugated to one or more effector molecules, cytosol localization elements, and/or subcellular compartment localization signal elements by any suitable technique, including those described further below to form the construct, which constructs can be used for therapeutic and/or diagnostic purposes.

Recombinant IL-13 is commercially available from a number of sources (e.g., R&D Systems, Minneapolis, MN, and Sanofi Bio-Industries, Inc., Tervose, PA). Alternatively, a gene or cDNA encoding IL-13 may be cloned into a plasmid or other expression vector and expressed in any of a number of expression systems according to methods well known to those of skill in the art. Methods of cloning and expressing IL-13 and the nucleic acid sequence for IL-13 are well known (see, e.g., Minty et al. (1993) and Mckenzie (1987)). Specific IL-13 mutants are also known and described in U.S. Pat. No. 6,630,576 (Debinski) and 6,884,603 (Debinski et al.), which are incorporated by reference herein. In some embodiments, the IL-13 mutant is IL-13.E13K, which has an amino acid residue at position 13 substituted for lysine. Other IL-13 mutants useful in the present invention include, but are not limited to, IL-13.R66D, IL-13.S69D, and IL-13.K105R. See Van Nguyen et al., Neuro-Oncology 14 (10): 1239-1253 (2012). Any mutant or combination of mutants may be used.

One of skill in the art will appreciate that analogues or fragments of IL-13 or IL-13 mutants can also specifically bind to IL-13RA2. For example, conservative substitutions of residues (e.g., a serine for an alanine or an aspartic acid for a glutamic acid) comprising native IL-13 may provide IL-13 analogues that also specifically bind to the IL-13 receptor. Thus, the terms “IL-13” or “IL-13 mutant” when used in reference to a targeting molecule, also includes fragments, analogues or peptide mimetics of IL-13 or IL-13 mutants that also specifically bind to the IL-13 receptor. Further discussion of IL-13 as contemplated by the present invention can be found in U.S. Pat. No. 5,328,984 (Pastan et al.), 5,614,191 (Puri et al.), 5,919,456 (Puri et al.), 6,296,843 (Debinski), 6,428,788 (Debinski et al.), 6,518,061 (Puri et al.), 6,576,232 (Debinski et al.), 6,630,576 (Debinski), 6,884,603 (Debinski et al.) and 8,362,207 (Debinski et al.).

Protein portions of the constructs may be produced using methods known in the art, e.g., bacterial expression, prokaryotic or eukaryotic expression, etc. See, e.g., U.S. Pat. No. 7,381,408 to Mezo et al.; U.S. Pat. No. 7,655,413 to Butt et al.; and U.S. Pat. No. 8,603,807 to Reed. In some embodiments, protein components may be produced by bacterial expression and/or by eukaryotic expression. For example, glycosylated IL-13.E13K-Fc-eA5-Cys can be produced using a baculovirus expression system in insect cells. In some embodiments, nucleic acids for expression of the protein components are codon optimized for the expression system (e.g. codon optimized for insect cells).

Targeting proteins as described herein may be coupled to or conjugated to a linker, another targeting protein and/or an effector molecule such as a diagnostic and/or therapeutic agent in accordance with any of a variety of techniques, such as those employed in the production of immunoconjugates. See, e.g., U.S. Pat. No. 6,949,245 to Sliwkowski.

In some embodiments, the construct is internalized in response to target protein binding. For example, EphA2 is over-expressed in a majority of patients with GBM and its ligand induces a receptor-mediated internalization once it binds the receptor (Walker-Daniels et al. (2002) Mol. Cancer Res. 1:79-87). The latter may be used for, e.g., recombinant bacterial toxin-containing cytotoxins to exert anti-tumor action (Debinski (2002) Molecular “Targeting of Brain Tumors with Cytotoxin,” In: Chimeric Toxins (Lorberboum-Galski & Lazarovici, eds., Harwood Academic Publishers) pp. 222-246; Debinski (2002) Cancer Invest. 20:801-809; Debinski (2002) Cancer Invest. 20:801-809). In addition, the IL-13RA2 receptor ligand is internalized through receptor mediated endocytosis. See also U.S. Pat. No. 8,362,207 to Debinski et al.

Chemotherapeutic agents useful as effectors include those described above. Small molecule toxins, such as a calicheamicin, a maytansine (see U.S. Pat. No. 5,208,020), a trichothene, and CC 1065 are also contemplated herein as effectors. In some embodiments, Pseudomonas exotoxins are used as effectors (see U.S. Pat. No. 5,328,984 to Pastan et al.). Enzymatically active toxins and fragments thereof which can be used as effectors include diphtheria A chain, nonbinding active fragments of diphtheria toxin, exotoxin A chain (from Pseudomonas aeruginosa), ricin A chain, abrin A chain (from Corrybacterium typhimuriae), modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin and the tricothecenes. See, for example, WO 93/21232.

Effectors may also include a compound with nucleolytic activity (e.g., a ribonuclease or a DNA endonuclease such as a deoxyribonuclease; DNase).

A variety of radioactive isotopes or radionuclides are available for the production of radioconjugated constructs as described above.

The linker may or may not be a peptide. Non-peptide linkers may include aliphatic hydrocarbon linkers such as an alkyl, alkyenyl or alkynyl, optionally including one or more functional groups suitable for covalent attachment of ligands, localization elements and/or effectors.

In some embodiments, conjugates of a targeting protein, linker and/or therapeutic agents or detectable groups may be made using a variety of bi-functional protein coupling agents such as N-succinimidyl-3-(2-pyridyldithiol) propionate (SPDP), succinimidyl-4-(N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin conjugate can be prepared as described in Vitetta et al. (1987) Science 238:1098. Carbon-14-labeled 1-isothiocyanatobenzyl-3-methyldiethylene triaminepentaacetic acid (MX-DTPA) is an exemplary chelating agent for conjugation of radionucleotide to the targeting peptide. See WO 94/11026. The linker may be a “cleavable linker” facilitating release of the cytotoxic drug in the cell. For example, an acid-labile linker, peptidase-sensitive linker, dimethyl linker or disulfide-containing linker (Chari et al. (1992) Cancer Res. 52:127-131) may be used.

In some embodiments, the linker may comprise an Fc domain or fragment thereof that is attached, directly or indirectly (for example, through a chemical spacer), to a ligand, localization element and/or effector molecule. The terms, “Fc”, “Fc domain” or “Fc fragment,” encompass native and altered forms of polypeptides derived from the Fc region of an antibody that are bound by an Fc receptor. In some embodiments, the Fc domain is derived from a human antibody (i.e., “human” Fc). The Fc domain normally has at least two heavy chain constant region domains (CH2 and CH3).

Forms of such Fc domains containing the hinge region that promotes dimerization are also included. One suitable Fc fragment, described in PCT applications WO 2005/047334 A1 and in WO 2004/074455 A2, is a single chain polypeptide extending from the N-terminal hinge region to the native C-terminus.

Also contemplated are altered forms of Fc fragments, for example, having improved serum half-life, altered effector functions, altered spatial orientation, and the like. The alteration of the Fc fragment can be achieved using any genetic engineering techniques known in the art. In some embodiments, the Fc domain is linked to more than one, for example, two, three or four, effector molecules.

Conjugation of the Fc domain may be performed using methods known in the art. See, e.g., U.S. 2010/0209424 to Roopenian et al. and U.S. 2012/0039880 to Yan et al., which are incorporated by reference herein. For example, a fusion protein including the ligand, localization signal element and/or effector may be made by recombinant techniques or peptide synthesis, which may also subsequently include covalent coupling of polypeptides, linkers and/or non-peptide effector(s).

In some embodiments, the Fc domain of the Fc fusion protein is a human Fc domain. The Fc domain may be from immunoglobulin G (IgG), IgA, IgE or IgM. In one embodiment, the Fc domain is from IgG, and may be from any of the subclasses of IgG. For example, in humans, there are four subclasses of IgG: IgG1; IgG2; IgG3; and IgG4. In some embodiments, the Fc domain is that of human IgG1.

In some embodiments, the Fc domain may include an antibody-dependent cellular cytotoxicity (ADCC) activating domain and/or a complement-dependent cytotoxicity (CDC) activating domain. Such domains may be useful in engaging and/or activating immune cells to attack the targeted cancer cells and add to their cytotoxic potency. See, e.g., Di Gaetano et al., Complement Activation Determines the Therapeutic Activity of Rituximab In Vivo, J Immunol 171:1581-87, 2003; U.S. Pat. No. 7,829,084 to Ledbetter et al.

In some embodiments, the effector molecule may be a Pseudomonas exotoxin or Diphtheria toxin. (U.S. Pat. No. 5,328,984 to Pastan et al. and U.S. Pat. No. 6,296,843 to Debinski). Pseudomonas exotoxins include, but are not limited to, Pseudomonas exotoxin A (PE). The Pseudomonas exotoxin can be modified such that it substantially lacks domain Ia, and Pseudomonas exotoxins may further include PE38QQR and PE4E. Diphtheria toxins include DT390, a diphtheria toxin in which the native binding domain is eliminated.

It will be appreciated that the targeting proteins and/or effector molecules can be connected to either of the amino terminus, or the carboxyl terminus, of a polypeptide linker, in addition to an internal amino acid (such as a cysteine).

### Pharmaceutical Formulations and Methods.

The constructs, conjugates, and/or compositions thereof described herein may be formulated for administration in a pharmaceutical carrier in accordance with known techniques. See, e.g., Remington, The Science and Practice of Pharmacy (9th Ed. 1995). In the manufacture of a pharmaceutical formulation according to the invention, the construct(s) (including the physiologically acceptable salts thereof) is typically admixed with, inter alia, an acceptable carrier. The carrier must, of course, be acceptable in the sense of being compatible with any other ingredients in the formulation and must not be unduly deleterious to the patient. The carrier may be a solid or a liquid, or both, and is preferably formulated with the construct(s) as a unit-dose formulation, for example, a tablet, which may contain from 0.01 or 0.5% to 95% or 99% by weight of the active construct. One or more active constructs may be incorporated in the formulations of the invention, which may be prepared by any of the well-known techniques of pharmacy comprising admixing the components, optionally including one or more accessory ingredients.

The formulations of the invention include those suitable for oral, rectal, topical, buccal (e.g., sub-lingual), vaginal, parenteral (e.g., subcutaneous, intramuscular, intradermal, or intravenous), topical (i.e., both skin and mucosal surfaces, including airway surfaces) and transdermal administration, although the most suitable route in any given case will depend on the nature and severity of the condition being treated and on the nature of the particular active construct which is being used.

Particular routes of parenteral administration include intrathecal injection (also for brain tumors spread locally to meninges), including directly into the tumor or a tumor resection cavity, and intraventricular injection into a ventricle of the brain.

Constructs and compositions may be administered by intratumor injection (including tumors in any region such as tumors of the breast and/or brain).

Particular routes of parenteral administration for breast cancer treatment include intravenous administration such as injection into a vein or venous catheter into the breast tissue or that leads into the breast tissue. In some embodiments, the active agent or construct is administered directly into the breast tissue and/or other regions of a subject where a breast cancer has metastasized. In some embodiments, the construct is administered to the subject through a central venous catheter (CVC; e.g., central venous line or central venous access device). In some embodiments, the CVC is a peripherally inserted central catheter (PICC line) or a tunneled CVC. In some embodiments, the construct is administered to the subject through a subcutaneous implanted port. In some embodiments, the intravenous administration may occur over about 1 minute to about 60 minutes (e.g., about 1, 2, 3, 4, 5, 10, 15, 20, 25, 30, 35, 40, 45, 50, 55, or about 60 minutes).

Formulations of the present invention suitable for parenteral administration comprise sterile aqueous and non-aqueous injection solutions of the active construct, which preparations are preferably isotonic with the blood of the intended recipient. These preparations may contain anti-oxidants, buffers, bacteriostats and solutes that render the formulation isotonic with the blood of the intended recipient. Aqueous and non-aqueous sterile suspensions may include suspending agents and thickening agents. The formulations may be presented in unit dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. For example, in one aspect of the present invention, there is provided an injectable or infusable, stable, sterile composition comprising an active construct or composition in a unit dosage form in a sealed container. The construct or composition is provided in the form of a lyophilizate that is capable of being reconstituted with a suitable pharmaceutically acceptable carrier to form a liquid composition suitable for administration thereof into a subject. The unit dosage form typically comprises from about 10 mg to about 10 grams of the construct or composition. When the construct or composition is substantially water-insoluble, a sufficient amount of emulsifying agent that is physiologically acceptable may be employed in sufficient quantity to emulsify the construct or composition in an aqueous carrier. One such useful emulsifying agent is phosphatidyl choline.

Further, the present invention provides liposomal formulations of the constructs disclosed herein and compositions thereof. The technology for forming liposomal suspensions is well known in the art. When the construct or composition thereof is an aqueous-soluble composition, using conventional liposome technology, the same may be incorporated into lipid vesicles. In such an instance, due to the water solubility of the construct or composition, the construct or composition will be substantially entrained within the hydrophilic center or core of the liposomes. The lipid layer employed may be of any conventional composition and may either contain cholesterol or may be cholesterol-free. When the construct or composition of interest is water-insoluble, again employing conventional liposome formation technology, the composition may be substantially entrained within the hydrophobic lipid bilayer that forms the structure of the liposome. In either instance, the liposomes that are produced may be reduced in size, as through the use of standard sonication and homogenization techniques.

Liposomal formulations containing the constructs disclosed herein or compositions thereof (e.g., multi-valent conjugates), may be lyophilized to produce a lyophilizate, which may be reconstituted with a pharmaceutically acceptable carrier, such as water, to regenerate a liposomal suspension. Examples of liposomal formulations that can be used include the neutral lipid 1,2-dioleoyl-sn-glycero-3-phosphatidylcholine (DPOC). See, e.g., Landen Jr. et al. (2005) Cancer Res. 65:6910-6918.

Other pharmaceutical compositions may be prepared from the water-insoluble constructs disclosed herein, or compositions thereof, such as aqueous base emulsions. In such an instance, the composition will contain a sufficient amount of pharmaceutically acceptable emulsifying agent to emulsify the desired amount of the construct or composition thereof. Particularly useful emulsifying agents include phosphatidyl cholines, and lecithin.

In addition to constructs, the pharmaceutical compositions may contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the compositions may contain microbial preservatives. Useful microbial preservatives include methylparaben, propylparaben, and benzyl alcohol. The microbial preservative is typically employed when the formulation is placed in a vial designed for multidose use. Of course, as indicated, the pharmaceutical compositions of the present invention may be lyophilized using techniques well-known in the art.

The therapeutically effective dosage could vary somewhat from construct to construct, and patient to patient, and will depend upon factors such as the age and condition of the patient and the route of delivery. Such dosages can be determined in accordance with routine pharmacological procedures known to those skilled in the art.

Particular pharmaceutical compositions comprising a construct of the present invention suitable for administration may include pH-neutral (e.g., from pH 6 or 6.5 to pH 7.5 or 8), low molarity saline solutions, for example, phosphate buffered saline (PBS) (such as Dulbecco's PBS pH 7.2).

As a general proposition, the initial pharmaceutically effective amount of the active construct administered parenterally will be in the range of about 0.1 to 50 mg/kg of patient body weight per day, with the typical initial range used being 0.3 to 20 mg/kg/day, more preferably 0.3 to 15 mg/kg/day. The desired dosage can be delivered by a single bolus administration, by multiple bolus administrations, or by continuous infusion administration of active construct, depending on the pattern of pharmacokinetic decay that the practitioner wishes to achieve.

The construct(s) may be suitably administered to the patient at one time or over a series of treatments. Depending on the type and severity of the disease, about 1 μg/kg to 15 mg/kg (e.g. 0.1-20 mg/kg) of active construct(s) is an initial candidate dosage for administration to the patient, whether, for example, by one or more separate administrations, or by continuous infusion. A typical daily dosage might range from about 0.1, 0.5, 1, 10 or 100 μg/kg up to 100, 200 or 500 mg/kg, or more, depending on the factors mentioned above. For repeated administrations over several days or longer, depending on the condition, the treatment is sustained until a desired suppression of disease symptoms occurs. A more particular dosage of the active construct will be in the range from about 0.05 mg/kg to about 20 mg/kg, for example from about 0.05 mg/kg, 0.25 mg/kg, 0.5 mg/kg, or 1.0 mg/kg, to about 5 mg/kg, 10 mg/kg, 15 mg/kg, or 20 mg/kg. Thus, one or more doses of about 0.5 mg/kg, 2.0 mg/kg, 4.0 mg/kg, 10 mg/kg, 15 mg/kg, or 20 mg/kg (or any combination thereof) may be administered to the patient.

Such doses may be administered intermittently, e.g., every week or every three weeks. An initial higher loading dose, followed by one or more lower doses may be administered. An exemplary dosing regimen comprises administering an initial loading dose of about 0.5 to 15 mg/kg, followed by a weekly maintenance dose of about 0.5 to, 5, 10, 15, or 20 mg/kg of the active construct. However, other dosage regimens may be useful. The progress of this therapy is easily monitored by conventional techniques and assays.

Subjects treated by the methods of the present invention can also be administered one or more additional therapeutic agents. See U.S. Pat. No. 5,677,178. Chemotherapeutic agents may be administered by methods well known to the skilled practitioner, including systemically, direct injection into the cancer, or by localization at the site of the cancer by associating the desired chemotherapeutic agent with an appropriate slow release material or intra-arterial perfusing of the tumor. The preferred dose may be chosen by the practitioner based on the nature of the cancer to be treated, and other factors routinely considered in administering. See, e.g., U.S. Pat. No. 7,078,030.

In some embodiments, the active agent or construct is administered directly into the brain (i.e., within the blood brain barrier) and/or other portions of the central nervous system of a subject. In some embodiments, the active agent is administered to the subject intra-cerebrally. In some embodiments, the active agent is administered to the subject by intracerebroventricular infusion. In some embodiments, the active agent is administered by intrathecal delivery. In some embodiments, the active agent is administered by convection-enhanced delivery.

Convection-enhanced delivery (CED) is the continuous injection under positive pressure of a fluid containing a therapeutic agent. In the central nervous system (CNS), this delivery technique circumvents the blood-brain barrier in delivering agents. See, e.g., U.S. 2005/0002918 to Strauss et al.; U.S. 2012/0041394 to Haider et al.; U.S. 2012/0209110 to Bankiewicz et al. CED uses a fluid pressure gradient established at the tip of an infusion catheter and bulk flow to propagate substances within the extracellular fluid space. CED allows the extracellularly-infused material to further propagate via the perivascular spaces and the rhythmic contractions of blood vessels acting as an efficient motive force for the infusate. As a result, a higher concentration of drug can be distributed more evenly over a larger area of targeted tissue than would be seen with a simple injection. CED has been clinically tested in the fields of neurodegenerative diseases and neurooncology, and is useful in a broad field of applications, such as the delivery of small molecules, macromolecules, viral particles, magnetic nanoparticles, and liposomes. In some embodiments, the CED includes the use of reflux-preventing catheter(s). In some embodiments, the CED includes real-time MRI monitoring.

With administration by continuous infusion such as CED, dosages of the construct for administration may be, for example, from 0.1 or 0.5 μg/mL to 20 μg/mL, for example, from 1, 2 or 3 μg/mL to 5, 8, 10 or 15 μg/mL.

In some embodiments, the construct is administered in combination with radiation therapy. In some embodiments, the construct is administered in combination with surgery to remove at least some of the cancerous tissue. In some embodiments, the construct is administered in combination with another, different chemotherapy agent.

Radiation therapy may include, e.g., external beam radiotherapy, which may be at any suitable dose (e.g., 20 to 70 Gy or more per tumor, typically delivered over a fractionated schedule).

Pharmaceutical compositions containing a targeting construct without an effector may be administered to subjects as blocking reagents, in like manner as described in Abrams et al., U.S. Pat. No. RE38,008, in conjunction with the administration of a targeting construct coupled to an effector such as a therapeutic group.

The targeting construct coupled to a detectible group may also be used in vitro as histological reagents on tissue samples, where binding of, e.g., the EphA2, EphA3, EphB2, or IL-13RA2 receptor is indicative of cancer tissue in the tissue sample.

Having described the present invention, the same will be explained in greater detail in the following examples, which are included herein for illustration purposes only, and which are not intended to be limiting to the invention.

### EXAMPLES

### Example 1: Development of a QUAD Targeting Construct

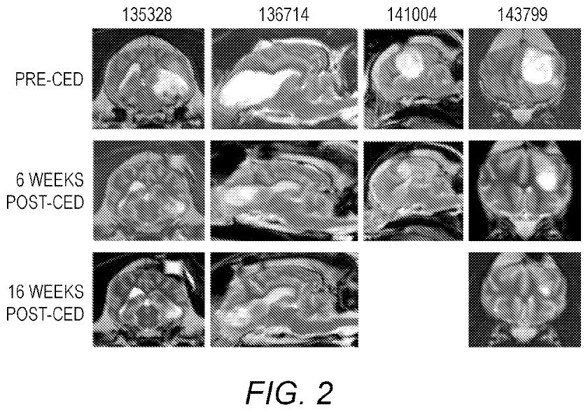

In first-of-a-kind approach, we performed Phase I clinical trial in dogs with gliomas, an excellent clinical surrogate of human disease, using a cocktail of cytotoxins targeting IL-13RA2 and EphA2 receptors. The cocktail was given loco-regionally through convection-enhanced delivery (CED) using anti-reflux catheters and real-time MRI monitoring of drug distribution. We were able to infuse not only enhancing, but also non-enhancing tumor, which contains infiltrating tumors cells. We observed prominent anti-tumor responses, including several near complete regressions, prolongation of survival and excellent quality of life in this dose-finding trial, although we did not achieve dose-limiting toxicity. A majority of dogs responded with >50% of tumor regression while we already observed 4/17 dogs with >95% tumor shrinkage and several long-term survivors (FIG. 2). See also Rossmeisl et al., Phase I trial of convection-enhanced delivery of IL13RA2 and EPHA2 receptor targeted cytotoxins in dogs with spontaneous intracranial gliomas. Neuro Oncol. 23 (3): 422-434 (2021).

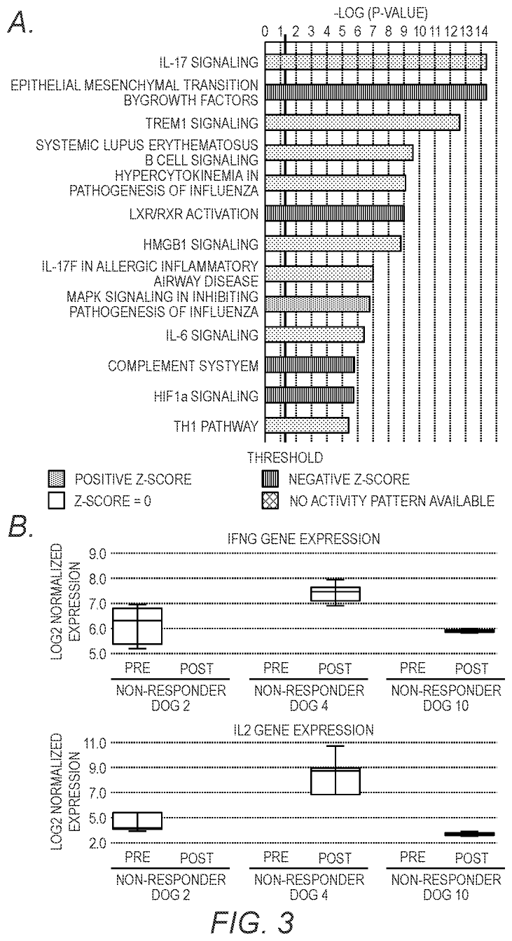

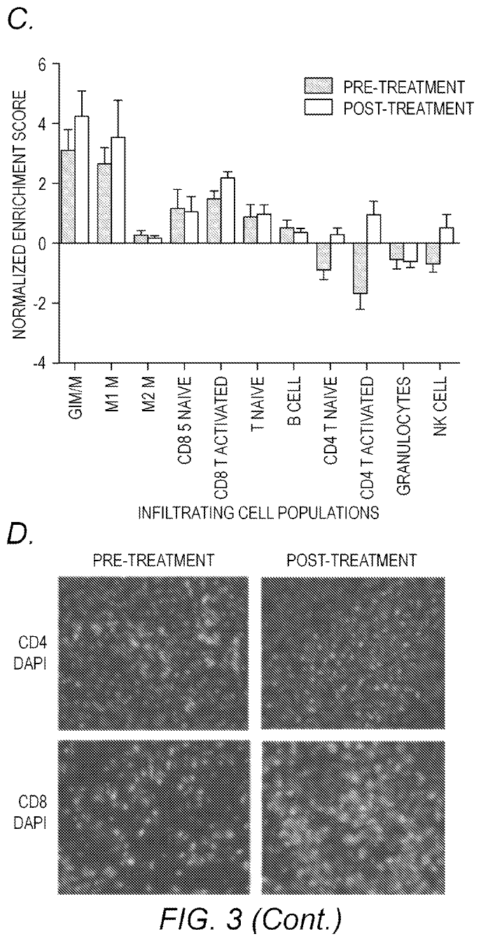

Using a canine immuno-oncology panel (Nanostring IO, Seattle, WA, USA), we have performed preliminary comparative gene expression analyses on pre- and post-treatment canine glioma samples from three dogs treated in our Phase I cocktail of cytotoxins trial (dogs 2, 4, and 10 in Rossmeisl et al., supra). The treatment resulted in activation of damage associated molecular pattern recognition pathways (FIG. 3A), upregulation of genes associated with cellular immunity and cytokine and chemokine signaling networks (FIG. 3B), as well as immune effector cell infiltration within tumors (FIG. 3C, D). Increased Th1 response cytokine gene expression was more robust in responder dogs and cytokine gene expression changes paralleled the serum cytokine analyses reported in the Phase I trial. Collectively, these data further support our hypothesis that the cytotoxin treatment induces immunogenic cell death and an in situ tumor vaccination type of effect.

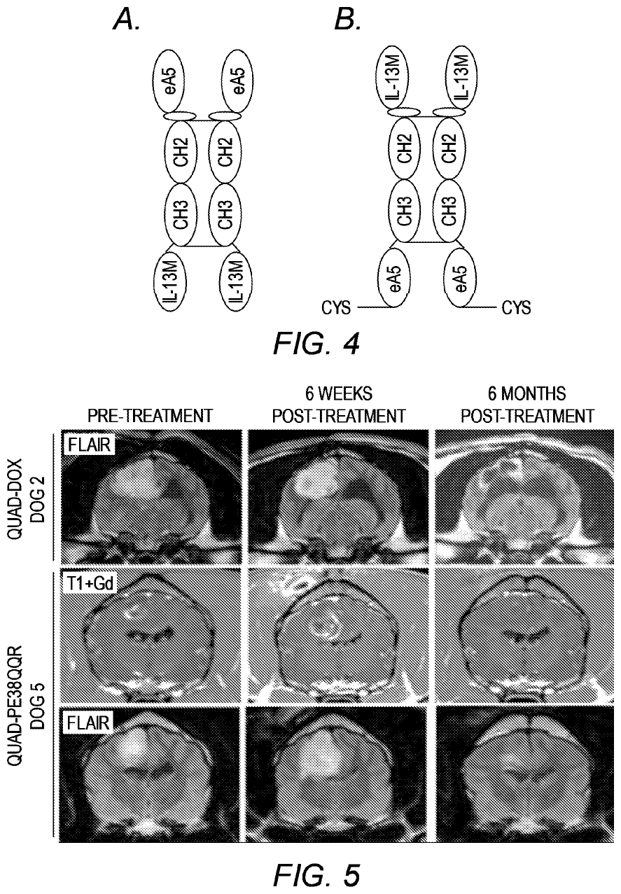

Encouraged by these exceptional results, in our next step we pursued the idea of targeting all four receptors with one pharmaceutical compound. To this end, we designed, produced and tested a multivalent ligand binding to all four receptors of our interest, termed QUAD, including murine and canine receptors. The molecule of QUAD 1 (FIG. 4, Panel A) was composed of (i) ephrin A5 (eA), which binds to EphA2, EphA3 and EphB2 receptors, (ii) an Fc region of an IgG1, and (iii) a mutated interleukin 13, IL-13.E13K, which binds IL-13RA2 [eA5-Fc-IL-13.E13K]. This protein chain was obtained frequently in a doublet seen on the SDS-PAGE, but changing the order of the ligand in a molecule solved this issue (QUAD 2). Indeed, IL-13.E13K-Fc-eA5 construct has reproducibly generated a single band of expected size ~150 kDa QUAD (e.g., FIG. 7B) (see Sharma et al., Multi-receptor targeting of glioblastoma. Neuro-Oncology Advances 2 (1), 1-11, 2020; Sharma et al., Drug Conjugates for Targeting Eph Receptors in Glioblastoma. Pharmaceuticals (Basel). 2020 Apr. 23; 13 (4): 77) at good yields.

We also started using High Five cells with an engineered ability to produce higher yields of recombinant proteins. High Five (H5) cells in suspension cultures produced up to 4.2 mg/L of media of the recombinant proteins. Insect cell-produced proteins have been approved for clinical use for both human and veterinary use. The QUAD can be purified to homogeneity and the ELISA assay demonstrated that all the four targeted receptors: IL-13RA2, EphhA2, EphA3, and EphB2 bind so generated QUAD, which is not a trivial achievement in a multivalent recombinant protein. In addition, we also made another version of QUAD, QUAD-Cys or QUAD 3, with an intent of having free thiol groups available for conjugation to drugs/labels, IL-13.E13K-Fc-eA5-Cys (FIG. 4, Panel B). This construct can be efficiently produced and purified as a single band protein, and the QUAD-Cys also binds effectively to all of the four targeted receptors. Thus, we have established and improved conditions for the QUAD production with fully preserved functions of the ligands and its further conjugation to drugs/labels.

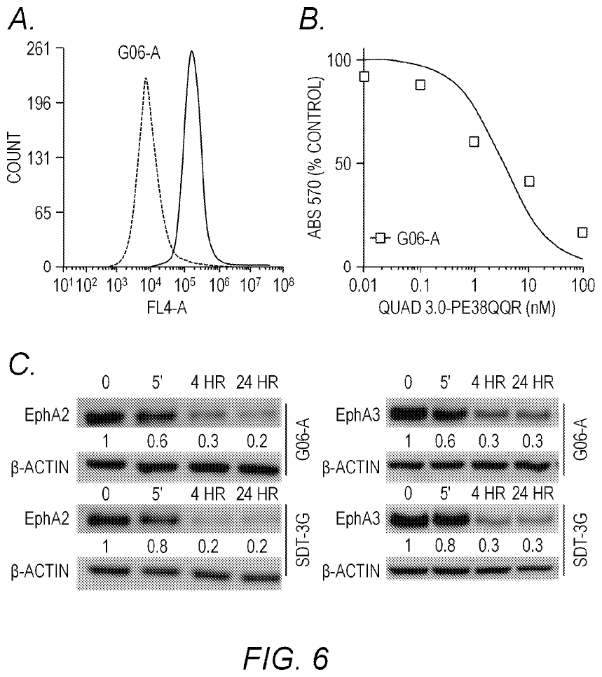

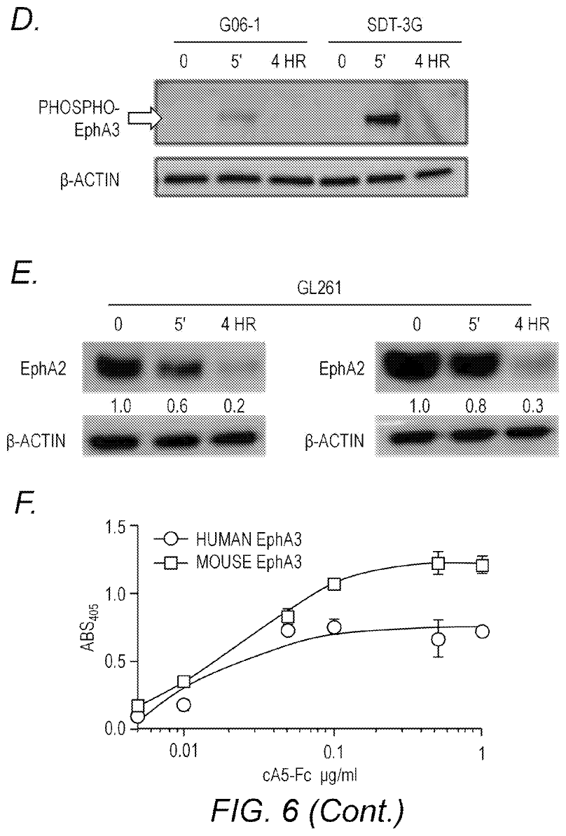

Next, we generated a conjugate between QUAD 3.0 and PE38QQR, which potently killed GBM cells (Sharma et al., Multi-receptor targeting of glioblastoma. Neuro-Oncology Advances 2 (1), 1-11, 2020). For example, the IC₅₀s were near 1.0 nM on U-251 and G48a, and 10 nM on BTCOE4795 GBM cells. These results directly demonstrated the feasibility and utility of the QUAD-drug approach. In addition, normal C57BL/6 mice were injected intracranially with a bolus of 0.1, 0.5 and 1.0 μg of QUAD 3.0-PE38QQR per mouse without any signs of toxicity. The QUAD was next conjugated to various derivatives of Doxorubicin (Dox) like WP1244, WP936 and WP1737, to generate single pharmaceutical drug conjugates. We have extensively characterized the conjugates as they retained their binding affinities towards the targeted receptors while demonstrating excellent killing activity on GBM cells (Sharma et al., Drug Conjugates for Targeting Eph Receptors in Glioblastoma. Pharmaceuticals (Basel). 2020 Apr. 23; 13 (4): 77). QUAD 3.0-WP1244 conjugate however, had the narrowest therapeutic window among the three conjugates tested and was dropped from further experimentation. As expected, the avid internalization of QUAD 3.0-WP936 was observed, and the cells exhibited readily detectable signals for the two individual components of the conjugate: QUAD and WP936, a fluorescent derivative of Dox. Thus, the QUAD 3.0-WP936 not only efficiently binds the targeted receptors in vitro but also recognizes live cells and it is internalized by them. Of high importance, QUAD 3.0-WP936 and QUAD 3.0-PE38QQR have already shown dramatic, long-lasting (>6 months) anti-tumor effects in dogs with spontaneous glioma without toxicity at a 1.6 μg/ml dose, half of the highest dose used in a cocktail trial (FIG. 5). These studies represent a proof-of-principle affirmation of our hypothesis that the QUAD protein can be a universal vector for effective drug deliveries. In addition, the modified IL-13 and eA5 ligands of human origin that are included in our constructs are fully reactive with the canine and mouse receptors, as reported previously for IL-13RA2 (Debinski et al., New agents for targeting of IL-13RA2 expressed in primary human and canine brain tumors. PLOS One. 2013 Oct. 16; 8 (10): e77719), and documented in further flow cytometry, cytotoxicity and functional assays for the canine/murine Eph receptors interaction with human eA5 (FIG. 6). This ascertains human relevance of all our animal studies, which is a strong point of our experimentations.

Our approach offers a unique opportunity to gain an increased access to tumor compartments of high resistance, or poor availability, to current treatment modalities. It also addresses the issues of high GBM tumor heterogeneity and immunosuppressive microenvironment.

### Example 2: Evaluation of Drug Conjugates

The QUAD was conjugated to various derivatives of Doxorubicin to generate single pharmaceutical compounds and these drug conjugates retained their binding affinities towards the targeted receptors while demonstrating excellent killing activity on GBM cells. One of the QUAD-Doxorubicin conjugates as well as the conjugate with a bacterial toxin derivative, PE38QQR, have already shown prominent anti-tumor responses in dogs with spontaneous glioma without toxicity. We have also found first evidence for immune system activation in targeted cytotoxins treated dogs based on blood and tissue analyses.

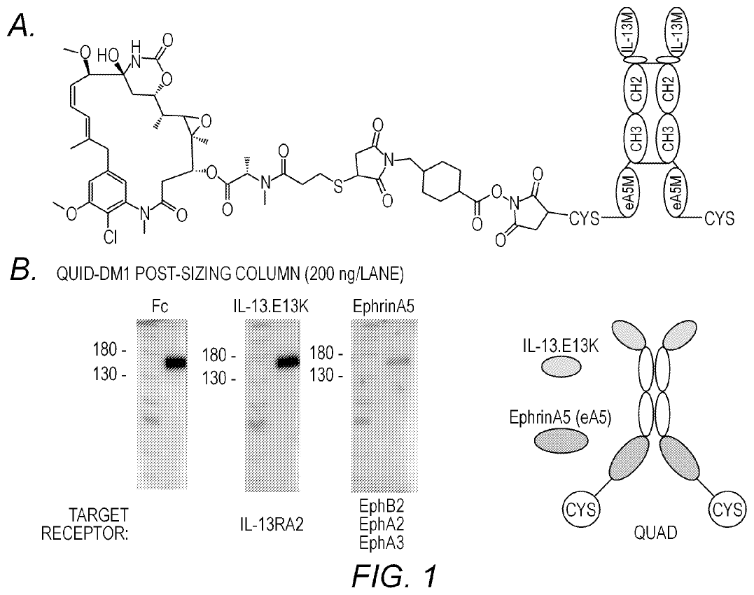

In further search for best options and most active QUAD-based drug conjugates, we decided to test other chemotherapeutics for conjugation with QUAD 3.0 or 3.1 (the 3.1 version has codon optimized for insect cells). We chose two drugs of very different modes of actions. One is Deruxtecan (DTX), a topoisomerase I inhibitor. The other is DMI, a microtubule disrupting agent (FIG. 7, Panel A). This agent is used for conjugation with Herceptin to produce Kadcyla, a highly effective anti-cancer drug (Park et al., I-SPY 2 Investigators. N Engl J Med. 2016 Jul. 7; 375 (1): 11-22). We have successfully conjugated the QUAD 3.0 protein to these two chemotherapeutics (e.g., FIG. 1, Panel A; DMI conjugate shown). Chemical conjugation of the QUAD 3.0/3.1 with DMI is performed as described previously (Debinski et al., An immunotoxin with increased activity and homogeneity produced by reducing the number of lysine residues in recombinant Pseudomonas exotoxin. Bioconj. Chem. 5:40-46, 1994; Debinski et al., (1995) Recombinant C242 F(ab′)-Pseudomonas exotoxin, but not the whole antibody-based immunotoxin, causes regression of a human colorectal carcinoma xenograft. Clin. Cancer Res. 1:1015-1022). QUAD 3.0/3.1 has a cysteine at the C-terminal end that provides a reactive thiol group for conjugation. Derivatized DM1 (DMI-SMCC, FIG. 7, Panel A) and DTX form stable thioether bonds through the cysteine residue in QUAD 3. The conjugates are purified from unconjugated counterparts using sizing exclusion chromatography with HiPrep™ 16/60 Sephacryl® S-200 HR (GE, Boston, MA) column.

Both conjugates were very potent in killing U-251 MG GBM cells (FIG. 7, Panel C). However, the IC₅₀ for the QUAD-DMI conjugate was in low femtomolar range, which is quite an extraordinary result as for a drug conjugate. Similar femtomolar range IC₅₀s were seen in A-172, BTCOE-4525 and BTCOE-4975 GBM cells (not shown). As shown in FIG. 7, Panel C, the activity of the QUAD-DMI conjugate was more than three logs of concentrations better from an unconjugated drug. QUAD-DMI was ~50 times more potent than all other QUAD-based drug conjugates tested: QUAD-WP936, QUAD-DTX, and also QUAD-PE38QQR (not shown). The QUAD-DMI conjugate was active not only on human GBM cells, but also on canine GBM cells (FIG. 7, Panel D). Moreover, the T98G GBM cells that do not express the IL-13RA25 and do not respond to single-targeted IL-13 based cytotoxins at all, were clearly impacted by QUAD-DMI (FIG. 7, Panel E). This is because T98G cells express some EphB2 and EphA3 receptors (not shown). Furthermore, the killing effect of QUAD-DMI was neutralized by an excess of unconjugated QUAD indicative of a competition for the binding sites between unconjugated QUAD and its DM1 conjugate (FIG. 7, Panel F). The same effect was seen on BTCOE4795 cells. Perhaps not surprisingly, as eA5 was previously shown to have tumor suppressive effect, the QUAD protein affected cell viability of U-251 GBM cells in vitro (FIG. 7, Panel G). This indicated for a possible dual anti-tumor action of QUAD conjugates. And finally, the QUAD-DMI conjugate was given either intracranially or intravenously in C57/BL6 non-tumor bearing mice (FIG. 7, Panels H, I). The highest doses used in these experiments that equaled the ones given IC with the other QUAD 3.0 conjugates (Sharma et al., Multi-receptor targeting of glioblastoma. Neuro-Oncology Advances 2 (1), 1-11, 2020; Sharma et al., Drug Conjugates for Targeting Eph Receptors in Glioblastoma. Pharmaceuticals (Basel). 2020 Apr. 23; 13 (4): 77) did not produce any signs of toxicity like changes in weight, neurological symptoms or IHC changes. In our most recent studies, we established that 4.0 μg of QUAD-DMI per mouse is a DLT for intracranial bolus injections. We have thus generated an extremely potent and safe drug conjugate, QUAD-DM1.

A multivalent targeted cytotoxic drug conjugate, QUAD-DMI (FIG. 1), has been engineered to target four receptors concomitantly [interleukin 13 receptor alpha 2 (IL-13RA2), EphA2, EphA3 and EphB2 receptors] that are overexpressed in patients with glioblastoma (GBM), the most prevalent primary brain tumor of dismal prognosis. The cytotoxic is a drug conjugate composed of targeting moieties and a microtubular inhibitor, DM1, which is used in an antibody drug conjugate named Kadcyla. The targeting moieties are as follows: modified for more specific binding towards the tumor associated receptor IL-13RA2, IL-13 (IL-13M), and ephrinA5 (eA5); both are located at the N-, and C-termini of an IgG1 scaffold (Fc), respectively, forming a multivalent protein, QUAD (FIG. 1). The recombinant product is produced in a single eukaryotic host cell (insect or mammalian, e.g., FS9 insect cells or HEK293 mammalian cells). QUAD-DM1 is a chemical conjugate of QUAD to DM1-SMCC. QUAD-DM1 is delivered using reflux preventing catheter(s) (CLEAR POINT Neuro, formerly MRI Interventions; e.g., IND #117959 or NCT02858895) under real-time MRI monitoring convection-enhanced delivery (CED).

This innovated approach to GBM treatment targets tumor cells, glioma stem-like cells (GSCs), neovasculature, infiltrating tumor cells, and TAMs, simultaneously, all expressing IL-13RA2, EphA2, EphA3 and EphB2 receptors, with one pharmaceutical compound, QUAD-DM1. The four receptors being targeted are present in abundance in virtually all GBM patients, meaning molecular pre-screening of patients may not be necessary for the proposed monotherapy. Furthermore, QUAD-DM1 is administered through convection-enhanced delivery (CED), which circumvents limitations posed by the blood-brain barrier (BBB) or blood-brain tumor barrier (BBTB).

### QUAD Production and Purification

High Five cells (High Five cells originated from the ovarian cells of the cabbage looper moth; Thermo Fisher, Waltham, MA) are dislodged from a 10-cm dish by sloughing. Viable cells are counted using a hemacytometer and trypan blue exclusion. 5×10⁵ cells/well are plated in a volume of 2 ml in a 6-well dish with serum free SFX-Insect cell culture media and incubated overnight at 27° C. without CO₂. The following day, the media is removed, and 1.8 ml of fresh media is added to the wells. 1-3 μg of QUAD plasmid (pMIB V5 His A, optimized for insect cells and synthesized by Genewiz) is prepared for transfection with Cellfectin according to the manufacturer's directions. Briefly, DNA is added to 100 μl of medium. In a separated tube, 4 μl of Cellectin is added to 100 μl of medium. The DNA and the Cellefectin tubes are gently combined and allowed to complex for 30 minutes at room temperature. The mixture is slowly added to the cells and incubated overnight at 27° C. without CO₂. The next day, the media (Cytiva, Marlborough, MA Thermo Fisher, Waltham, MA) is replaced and the cells are allowed to recover overnight. Positively transfected cells are selected with 500 μg/ml blasticidin. Positive selection is maintained with 50 μg/ml blasticidin. Cells are expanded into suspension culture at a density of 3×10⁵ cells/cm². After 3-4 days in culture, suspension cells are centrifuges at 1,000×g for 5 minutes to harvest the media containing the secreted QUAD protein.

QUAD containing media is filtered through a 0.22 μm filter prior to purification on a HiTrap Protein G HP (Cytiva) column using AKTA Pure FPLC system (Cytiva, Marlborough, MA). Column is first equilibrated with 10 column volumes (CV) of 20 mM sodium phosphate, pH 7.0 prior to loading QUAD containing media. After washing with 10 CV of 20 mM sodium phosphate, pH 7.0, protein is eluted with 0.1 M glycine-HCl, pH 2.7 in 1-ml fractions. The pH of the eluate is immediately adjusted to 7.0 by the addition of 200 μl IM TRIS-HCl, pH 8.0. Positive fractions (based on SDS-PAGE) are pooled, concentrated and buffer exchanged to Dulbecco's phosphate buffered saline, pH 7.2 containing 5 mM EDTA (D-PBS/EDTA) with a 30K MWCO centrifuge filter (Sartorius Vivaspin). QUAD is diluted to a concentration of 1 mg/ml and stored at −80° C. until use. The purity is verified by SDS-PAGE.

### QUAD Conjugation and Conjugate Purification

QUAD is conjugated to DMI at a 16x molar ratio. 10.85 μl of 5 mM DM1 in DMSO is added to 189.15 μl D-PBS/EDTA. This mixture is slowly added drop by drop to 200 μg of QUAD (in 200 μl), while stirring (130 rpm max). The mixture is incubated to room temperature for 60 minutes while stirring. Reaction is allowed to continue overnight at 4° C. The next day, the mixture is centrifuged at 10,000×g 5 for minutes to remove any precipitates, if present.

At least 1 mg of conjugated QUAD is concentrated using a 30K MWCO centrifuge filter prior to injection onto a HiPrep 16/60 Sephacryl S-300 HR column (Cytiva). Columns are run at a flow rate of 0.5 ml/min and 0.4 ml fractions are collected. Positive fractions are pooled, concentrated and stored at −80° C. The identity and purity of the conjugate is confirmed by Western blot (FIG. 1B).

The conjugate will be diluted to the final concentration with the formulation buffer and sterile filtered into final product container/closures (e.g., pharmaceutical grade 1.0 mL glass serum vials with rubber serum stoppers and aluminum crimp seals).

### Initial QUAD-DMI Conjugate Stability Studies

Methods: QUAD-DM1 is incubated in plasma from a GBM patient, a non-tumor patient (normal) or in PBS for up to 8 weeks in a volume of 100 μl at −80° C., −20° C., 40° C. and 27° C. (RT) and 37° C. At the designated time points, PBS containing 0.1% BSA is added and the mixture is filtered through a 0.22 μm filter. The QUADcyla is serially diluted and added to U-251 GBM cells in an MTT cell viability assay.

Results: QUAD-DMI maintained cell killing activity for at least 8 weeks after incubation in PBS, GBM patient plasma and normal patient plasma at −80° C., −20° C. and also at 4° C. When incubated at RT, QUAD-DMI began to lose killing activity after 1 week for the plasma-incubated samples. QUAD-DMI incubated in PBS at RT also retained most of its killing efficiency up to 8 weeks. After incubation at 37° C. for 4 weeks, the QUAD-DM1 in plasma lost 3 logs of killing activity while the PBS incubated sample lost only one log of activity. After 8 weeks at 37° C., all killing activity was lost in all conditions.

### Example 3: Preclinical Study

Chemical conjugation of the QUAD with DMI is performed as described previously. QUAD has a cysteine at the C-terminal end that provides a reactive thiol group for conjugation. Derivatized DM1 (DM1-SMCC) forms stable thioether bonds through the cysteine residue in QUAD. The conjugates are purified from unconjugated counterparts using sizing exclusion chromatography with HiPrep™ 16/60 Sephacryl® S-200 HR (GE, Boston, MA) column.

We demonstrate in FIG. 8 a dramatic response to QUAD-DMI with no toxicity, in a dog with spontaneous high-grade glioma. This was somewhat expected by us due to the described favorable properties of QUAD-DM1. It is possible that this case will turn into a CR as the remaining enhancing lesions on MRI after cytotoxins treatment usually undergo further dissolution with time.

### Example 4: Evaluation of QUAD Treatments on Breast Cancer and Breast Cancer Brain Metastasis

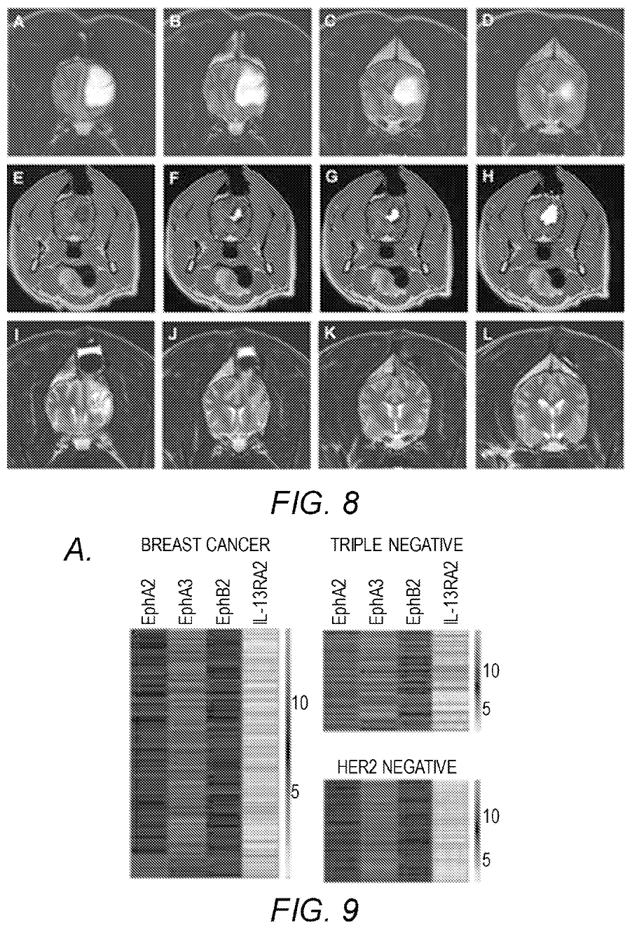

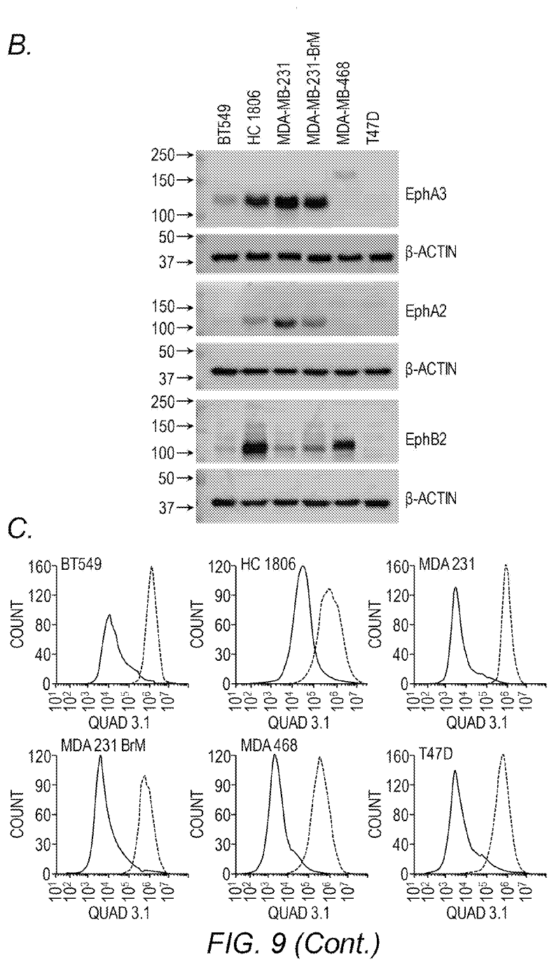

As triple-negative breast cancer (TNBC) remains an unmet need in medicine, we examined the presence of the four QUAD-targeted receptors in breast cancer cells and tissue micro-arrays, including involved lymph nodes, and in paired primary tumor-brain metastases, spanning two molecular subtypes of breast cancer: TNBC and HER2+. We found that the four targeted receptors are expressed in almost all breast cancer and related metastases to brain specimens, based on the expression levels for the genes and the gene products (FIG. 9, Panel A). We also identified several established breast cancer cell lines which expressed EphA3, EphA2, and EphB2 by western blot (FIG. 9, Panel B). Cell lysates were collected from sub-confluent cultures in RIPA buffer containing protease and phosphatase inhibitors (Sigma). Proteins were separated by 10% SDS-PAGE under reducing conditions and transferred to PVDF membranes. After blocking of non-specific interactions, primary antibodies were added and incubated overnight at 4° C. The antibodies used were EphA2 (see Wykosky, et al. Mol. Cancer Ther. 6 (12): 3208-3218 (2007)), EphA3 (MyBiosource) and EphB2 (R&D Systems). After multiple washes with PBS, secondary antibodies were added (Sigma). Chemiluminescent detection was performed, and bands were detected with an Amersham RGB600 imager. Equal loading of proteins was verified by probing for B-Actin (Sigma).

Flow cytometry was performed to analyze the binding of QUAD to human breast cancer cell lines (FIG. 9, Panel C). Cells were detached with Versene and washed once with PBS. 200,000 cells in PBS/1% BSA were aliquoted into tubes and incubated on ice for 1 hr to block non-specific sites. 2 μg QUAD protein or human Fc control was added to the tubes and incubated on ice for 2 hrs with occasional mixing. Cells were washed with PBS/1% BSA and secondary antibody was added (anti-human Alexa fluor 647, Invitrogen). Following 1 hr incubation on ice, cells were washed and fixed with formalin. Detection was performed on an Accuri6 flow cytometer (BD Biosciences) and data were analyzed with FCS Express (DeNovo Software).

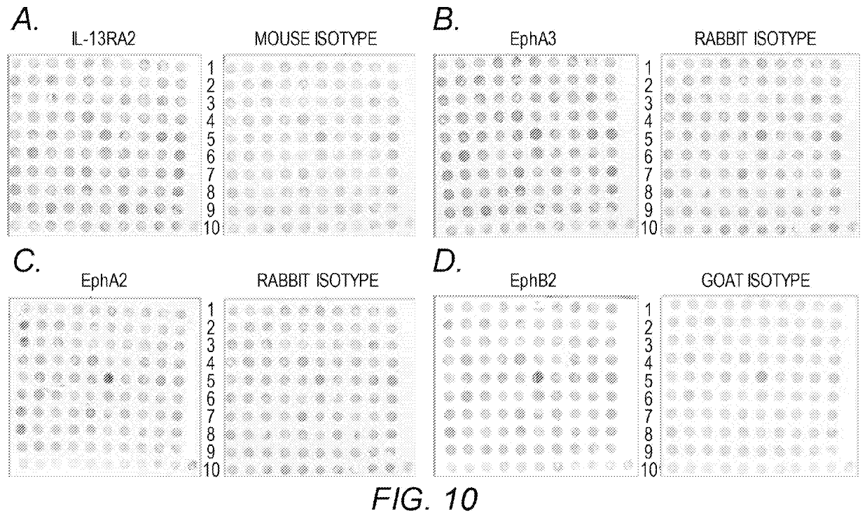

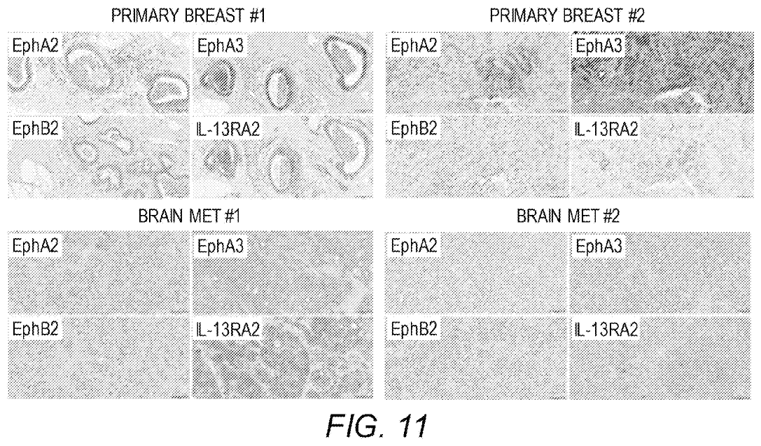

Immunohistochemistry (IHC) data from tissue microarrays (TMAs) from human breast cancer, lymph node metastases, and normal breast tissue was stained for the presence of IL-13RA2, EphA3, EphA2, and EphB2 proteins (FIG. 10). Staining was performed on US Biomax slides which were deparaffinized with xylene and rehydrated through ethanol. Endogenous peroxides were quenched and antigens were retrieved by heating in sodium citrate, pH 6.0. Non-specific binding was blocked with Superblock (Scytek) and slides were stained for IL-13RA2 (0.5 μg/ml; clone 1E10B9, see Debinski et al., PLOS One (2013))), EphA3 (1:200; Genetex NIN3), EphA2 (2.5 μg/ml; NovusBio), and EphB2 (2 μg/ml; R&D Systems). After overnight incubation at 4° C., slides were washed with PBS prior to application of secondary antibody (anti-rabbit polymer HRP, Vector Labs). Detection was performed with Nova Red (Vector Labs). Slides were counterstained with hematoxylin, dehydrated, cleared with xylene, and mounted with Permount. Slides were digitally scanned in the Virtual Microscope Core Lab of the WFU Comprehensive Cancer Center. In comparison to the normal tissue and isotype-stained control, these data showed that the IL-13RA2, EphA3, EphA2, and EphB2 proteins were prevalent in the breast cancer tissue and lymph node metastases. Similar IHC staining in primary breast cancer and brain metastases from two patients produced the same results and demonstrated the presence of these proteins in both tissues (FIG. 11). Histological scoring of IL-13RA2, EphA3, EphA2, and EphB2, estrogen receptor (ER), progesterone receptor (PR), and HER2 of ten patient matched breast cancer and its brain metastasis obtained from the patient's original pathology report following surgery is shown below in Table 1.

TABLE 1
Histological scoring in patient cancer samples.

| Patient | Breast cancer | IL-13RA2 | EphA3 | EphA2 | EphB2 | ER | PR | HER2 |
|---|---|---|---|---|---|---|---|---|
| 1 | Primary | 2+ | 2+ | 1+ | − | − | − | − |
| 1 | Brain metastasis | 2+ | 1+ | 1+ | − | − | − | − |
| 2 | Primary | − | 3+ | 1+ | − | + | + | + |
| 2 | Brain metastasis | 2+ | 1+ | 1+ | 1+ | + | + | + |
| 3 | Primary | − | 2+ | 1+ | − | − | − | + |
| 3 | Brain metastasis | 2+ | 1+ | 1+ | 1+ | − | − | + |
| 4 | Primary | 3+ | 3+ | 3+ | − | + | − | + |
| 4 | Brain metastasis | − | − | − | − | + | − | + |
| 5 | Primary | 2+ | 2+ | 1+ | − | + | − | + |
| 5 | Brain metastasis | 2+ | 2+ | 2+ | − | + | − | + |
| 6 | Primary | 2+ | 2+ | 2+ | 1+ | − | − | + |
| 6 | Brain metastasis | 1+ | 1+ | 1+ | − | − | − | + |
| 7 | Primary | 1+ | 1+ | 1+ | − | − | − | − |
| 7 | Brain metastasis | − | − | 3+ | 1+ | − | − | − |
| 8 | Primary | 1+ | 2+ | 2+ | 2+ | + | + | − |
| 8 | Brain metastasis | − | 3+ | 2+ | − | + | + | − |
| 9 | Primary | − | 3+ | 3+ | − | − | − | − |
| 9 | Brain metastasis | 1+ | 2+ | 2+ | − | − | − | − |
| 10 | Primary | 1+ | 1+ | − | − | − | − | − |
| 10 | Brain metastasis | 1+ | 2+ | − | − | − | − | − |

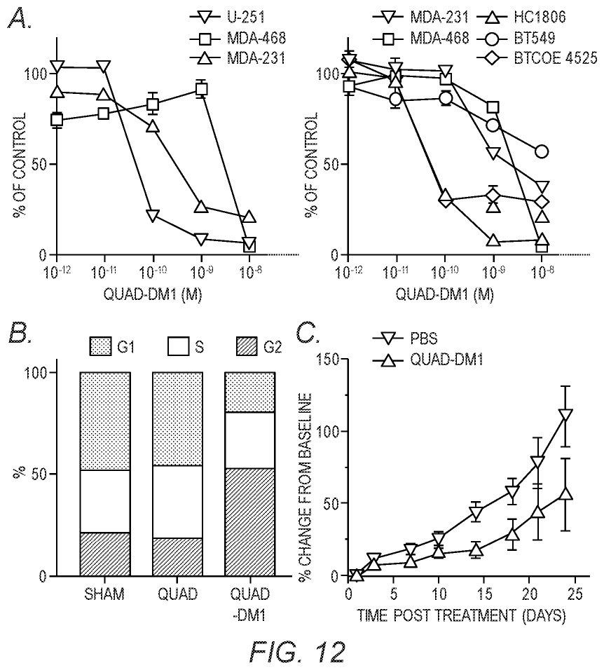

We tested QUAD-DMI on multiple human breast cancer cell lines and found that they were highly responsive to treatment (FIG. 12, Panel A). Specifically, the breast cancer cell lines like HC1806 (acantholytic squamous cell carcinoma), MDA-MB-468 (metastatic adenocarcinoma), MDA-MB-231 (adenocarcinoma), BT549 (ductal carcinoma), and the breast metastatic adenocarcinoma cell line MDA-MB-231-BRM cells are killed at up to picomolar concentrations of QUAD-DM1. Further cell staining and flow cytometry analysis showed that QUAD-DMI causes cell cycle arrest in G2 phase (FIG. 12, Panel B). Log phase MDA-MB-231 cells were serum starved for 24 hours, then normal growth media with 1 nM QUAD or human Fc (sham) was applied. After an additional 24 hours, the cells were collected and fixed with cold ethanol. Nuclei were stained with Propidium Iodide containing RNAse A (BD Biosciences). Cell cycle data was collected on a Canto II Flow Cytometer (BD Bioscience). Data was analyzed with FCS Express using multicycle DNA analysis and graphed in Prism GraphPad. Unconjugated QUAD (100 nM) neutralizes cell killing activity of QUAD-DM1.

MDA-MB-231 tumors growing in mammary pads of athymic mice responded significantly to QUAD-DMI given intravenously (FIG. 12, Panel C), indicating that QUAD recognizes murine receptors similarly to human. Nude mice were injected into the fourth mammary fat pad with 500,000 MDA-MB-231 cells in a volume of 100 μL. Tumors were measured with calipers twice per week and volume was calculated using the formula V=(W2×L)/2. Tumors were allowed to grow until they reached 100 cubic mm. Mice were randomized into 2 groups and either 240 μg of QUAD-DM-1 (12 mg/kg) or PBS was injected IV into the tail vein in a volume of 100 μL. Injections were continued weekly. Mice were euthanized on day 24. This data supports QUAD-DMI as a novel type of a multivalent drug conjugate for the IV treatment of breast cancer and its brain metastases, which is applicable, but not exclusively, to TNBC.

### Example 5: Further Evaluation of QUAD Treatments

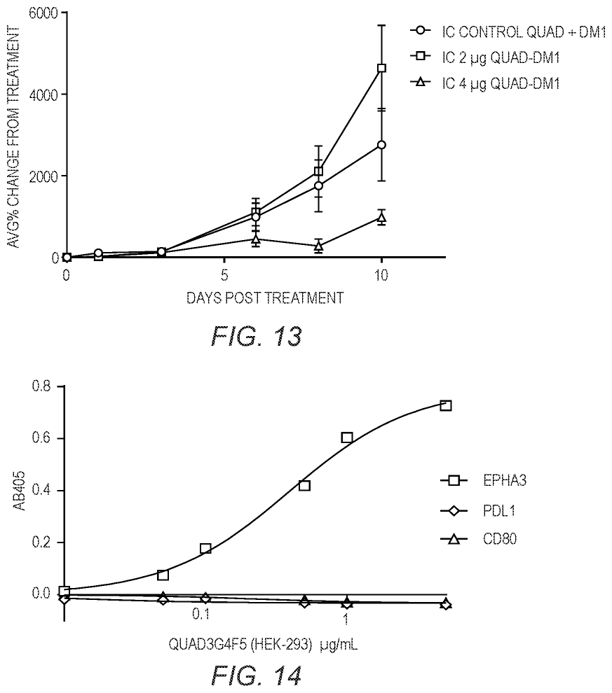

FIG. 13 presents data of intracranial (IC) treatment of MDA-MB-231-BrM tumors with

QUAD-DM1, 200,000 MDA-MB-231-BrM cells in 2 μL-IC implantation. Treatment started 7 days post tumor cells implantation (Day 0). Animals were imaged 3 times per week via IVIS imaging with bio-luminescence (photons per sec). Shown in FIG. 13 is the average percentage change in response to treatment. These data demonstrate that QUAD-DMI produced significant anti-tumor effect in mice with intracranial tumors of breast cancer brain metastasis origin.

FIG. 14 presents data on the binding specificity (ELISA assay) of HeK-293 cells-produced QUAD to: EphA3, PDL1, and CD80. QUAD binds only to the EphA3 receptor as expected. Hek-293 cells are human embryonic kidney cells. The same result was obtained with QUAD produced in Expi-293F cells that are HEK-293 cells adapted for transient transfection. These data demonstrate that QUAD-DMI binding is specific to the targeted receptors.

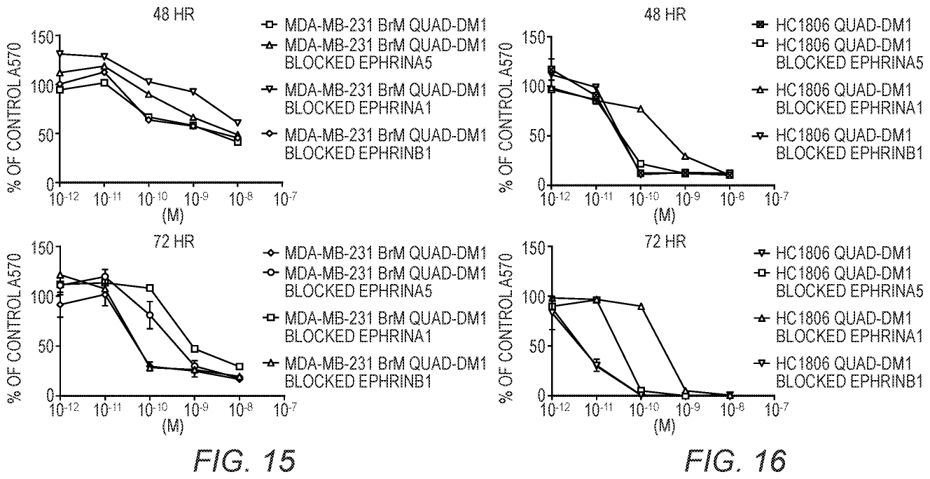

FIG. 15 presents data of MDA-MB-231 BrM cell killing by QUAD-DMI read at 48 and 72 hrs after adding QUAD-DM1. The cell killing by QUAD-DMI was partially blocked by the antibodies against individual receptor binding abilities of the QUAD ligand. These data demonstrate that the killing effect is dependent on the binding to the Eph receptors by eA5, eA1, and eB1 ephrin binding activity.

FIG. 16 presents data of HC1806-triple negative breast cancer cells (TNBC), with methods similar to that of the data in FIG. 15.

Bioluminescence imaging by IVIS. Intracranial tumors: Nu/nu mice were intracranially injected with 200,000 MDA-MB-231-BrM-luc-RFP cells via stereotaxic injection. 7 days after injection and after verification of tumor growth, mice were intracranially infused once with QUAD-DMI or unconjugated QUAD+DM1. Three times per week, mice were intraperitoneally injected with 150 mg/kg D-luciferin (Gold Biotechnology, St Louis MO) and imaged using the in vivo Imaging System (Perkin Elmer, Shelton, CT).

Using non-optimized dose of the conjugate, we observed significant tumor growth inhibition in the visual recoding of treatment of mice with IC tumors (data not shown). These results affirm that the set of IL-13RA2, EphA3, EphA2 and EphB2 receptors presents an excellent quartet of targetable receptors in peripheral and brain metastatic breast cancer.

In experiments with MDA-MB-231 BrM treated with QUAD-DMI, QUAD-DMI was found to be internalized by the treated cells (data not shown), which results in potent cell killing.

The foregoing examples are illustrative of the present invention and are not to be construed as limiting thereof. Although the invention has been described in detail with reference to preferred embodiments, variations and modifications exist within the scope and spirit of the invention as described and defined in the following claims.

1. A cancer targeting construct comprising a first end and a second end,
said first end having two IL-13 proteins, or two IL-13RA2 binding mutants or fragments of IL-13,
said second end having two eA5 proteins, or two EphA2, EphA3 and EphB2 binding mutants or fragments of eA5, and
an effector molecule coupled to said first end or said second end,
wherein said construct is a fusion protein and/or covalent conjugate, and
wherein the effector molecule comprises a maytansinoid or a maytansinoid analog.

2. The construct of claim 1, wherein the two eA5 proteins are mutant eA5 proteins and/or wherein the two IL-13 proteins are mutant IL-13 proteins.

3. The construct of claim 1, wherein the two eA5 proteins are fragments of eA5.

4. (canceled)

5. The construct of claim 1, wherein said eA5 and IL-13 proteins are glycosylated.

6. The construct of claim 1, wherein said construct comprises a fusion protein with a protein linker between said first end and said second end.

7. The construct of claim 6, wherein said first end is at the N-terminus of the fusion protein, and the second end is at the C-terminus of the fusion protein.

8. The construct of claim 6, wherein said protein linker comprises an ADCC and/or CDC activating domain.

9. The construct of claim 6, wherein said protein linker comprises an Fc fragment of human IgG1.

10. The construct of claim 1, wherein said effector molecule further comprises a detectable group.

11. A composition comprising the construct of claim 1 and a pharmaceutically acceptable carrier.

12. The composition of claim 11, wherein said carrier is sterile.

13. The composition of claim 11, wherein said carrier is a saline solution having a pH of from 6 to 8.

14. The composition of claim 11, wherein said composition is suitable for administration to a subject by infusion.

15. The composition of claim 14, wherein said composition is suitable for administration by continuous infusion of from 0.1 or 0.5 μg/mL to 20 μg/mL.

16. A nucleic acid that encodes the construct of claim 1, or a protein or peptide portion thereof.

17. A eukaryotic host cell that contains the nucleic acid of claim 16 and is configured to express the encoded peptide.

18. The eukaryotic host cell of claim 17, wherein said host cell is an insect expression cell.

19. A method of treating cancer in a subject in need thereof, comprising administering to said subject the construct of claim 1 in a treatment effective amount.

20. The method of claim 19, wherein said cancer is breast cancer, bladder cancer, pancreatic cancer, colorectal cancer, head and neck cancer, thyroid cancer, prostate cancer, melanoma, or glioma.

21.-22. (canceled)

23. The method of claim 19, wherein said administering comprises convention-enhanced delivery (CED).

24. The method of claim 19, wherein said construct is administered to the subject by continuous infusion of from 0.1 or 0.5 g/mL to 20 μg/mL.

25. A method of detecting EphA2, EphA3 and/or EphB2 expressing cells, comprising administering the construct of claim 1 to a cell or group of cells, wherein said construct comprises a detectable group, and detecting said detectable group.

26.-36. (canceled)

37. A method of treating breast cancer in a subject in need thereof, comprising administering to said subject the construct of claim 1 in a treatment effective amount.

38. The method of claim 37, wherein the breast cancer comprises squamous cell carcinoma, adenocarcinoma, or ductal carcinoma.

39. The method of claim 37, wherein the breast cancer is triple-negative breast cancer.

40. The method of claim 37, wherein the breast cancer is metastatic breast cancer.

41. The method of claim 40, wherein the metastatic breast cancer is metastasized to the bones, lungs, brain, and/or liver.

42. (canceled)

43. The method of claim 37, wherein said administering comprises intravenous injection, optionally wherein said administering comprises intravenous injection through a central venous catheter.

44. (canceled)

45. The method of claim 43, wherein the intravenous injection is carried out over about 1 minute to about 60 minutes.

46. The method of claim 37, wherein the administering comprises administering from about 0.1 mg/kg to about 15 mg/kg of the construct.
